(19) United States
(12) Patent Application Publication
(10) Pub. No.: US 2007/0207166 A1
(43) Pub. Date: Sep. 6, 2007

(54) Method of Using Adenoviral Vectors to Induce an Immune Response

(75) Inventors: Gary J. Nabel, Washington, DC (US); Cheng Cheng, Bethesda, MD (US); Wing-Pui Kong, Gaitersburg, MD (US); Jason G.D. Gall, Germantown, MD (US); C. Richter King, Washington, DC (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: GenVec, Inc., Gaithersburg, MD (US)

(21) Appl. No.: 11/578,096

(22) PCT Filed: Apr. 12, 2005
(86) PCT No.: PCT/US05/12291

§ 371 Date: Jan. 4, 2007

Related U.S. Application Data
(60) Provisional application No. 60/561,341, filed on Apr. 12, 2004.

(51) Int. Cl. A61K 39/12; C12N 15/861

(52) U.S. Cl. 424/199.1; 424/93.2; 435/456

(57) ABSTRACT

The invention provides a method of inducing an immune response against a human immunodeficiency virus (HIV) in a mammal. The method comprises administering to the mammal an adenoviral vector composition comprising one or more adenoviral vectors encoding two or more different HIV antigens, the production of which induces an immune response against HIV in the mammal. The invention also provides an adenoviral vector composition comprising four adenoviral vectors encoding an HIV clade A Env protein, an HIV clade B Env protein, an HIV clade C Env protein, and a fusion protein comprising an HIV clade B Gag protein and Pol protein, respectively.

### CROSS-REFERENCE TO RELATED APPLICATIONS

This patent application claims the benefit of U.S. Provisional Patent Application No. 60/561,341, filed Apr. 12, 2004.

### STATEMENT REGARDING FEDERALLY SPONSORED RESEARCH AND DEVELOPMENT

This invention was made in part with Government support under Cooperative Research and Development Agreement (CRADA) Number Al-1034, and amendments thereto, executed between GenVec, Inc. and the U.S. Public Health Service representing the National Institute of Allergy and Infectious Diseases. The Government may have certain rights in this invention.

### BACKGROUND OF THE INVENTION

The Centers for Disease Control and Prevention (CDC) estimate that in the United States, 850,000 to 950,000 people are living with HIV infection and approximately 25% are unaware of their infection (CDC, Morb. Mortal. Wkly. Rep., 52(47), 1145-8 (2003)). Worldwide, the rate of new HIV infections continues to increase at an unacceptably high level. Although new AIDS diagnoses and deaths have fallen significantly in developed countries since the advent of highly active antiretroviral therapy (HAART), in the developing world the HIV/AIDS epidemic continues to accelerate. The global impact of the epidemic is considerable. According to the Joint United Nations Programme on HIV/AIDS and the World Health Organization, as of the end of 2002, 40-42 million people were estimated to be living with HIV/AIDS, with 95% of the global total residing in the developing world (WHO, Treating 3 Million by 2005: The WHO Strategy, Geneva, Switzerland. p. 1-53 (2003), and UNAIDS, AIDS Epidemic Update December 2003). Worldwide there were an estimated 2.5-3.5 million deaths due to HIV/AIDS in 2003 (UNAIDS, AIDS Epidemic Update December 2003) and there have been as many as 30 million deaths as a result of HIV infection since the beginning of the epidemic (WHO, Treating 3 Million by 2005: The WHO Strategy, Geneva, Switzerland. p. 1-53 (2003)). Beyond the human tragedy of HIV/AIDS, the costs of the epidemic pose a significant impediment to the economic growth and Political stability of many countries. In developing countries and in segments of the U.S. population, anti-HIV therapies are frequently beyond financial reach. Accordingly, effective, low-cost tools for HIV prevention, such as a vaccine, are urgently needed to bring the HIV epidemic under control.

Delivery of proteins as therapeutics or for inducing an immune response in biologically relevant amounts has been an obstacle to drug and vaccine development for decades. One solution that has proven to be a successful alternative to traditional antigen delivery approaches is delivery of exogenous nucleic acid sequences for production of antigenic molecules in vivo. Gene transfer vectors ideally enter a wide variety of cell types, have the capacity to accept large nucleic acid sequences, are safe, and can be produced in quantities required for treating patients. Viral vectors have these advantageous properties and are used in a variety of protocols to treat or prevent biological disorders.

Despite their advantageous properties, widespread use of viral gene transfer vectors is hindered by several factors. In this regard, certain cells are not readily amenable to gene delivery by currently available viral vectors. For example, lymphocytes are impaired in the uptake of adenoviruses (Silver et al., Virology 165, 377-387 (1988); Horvath et al., J. Virology, 62(1), 341-345 (1988)).

The use of viral gene transfer vectors also is impeded by the immunogenicity of viral vectors. A majority of the U.S. population has been exposed to wild-type forms of many of the viruses currently under development as gene transfer vectors (e.g., adenovirus). As a result, much of the U.S. population has developed pre-existing immunity to certain virus-based gene transfer vectors. Such vectors are quickly cleared from the bloodstream, thereby reducing the effectiveness of the vector in delivering biologically relevant amounts of a gene product. Moreover, the immunogenicity of certain viral vectors prevents efficient repeat dosing, which can be advantageous for “boosting” the immune system against pathogens, and results in only a small fraction of a dose of the viral vector delivering its payload to host cells.

In addition, a major challenge in the design of viral vectors as HIV vaccines is to identify and target viral structures that are the critical determinants for protective humoral and cellular immune responses across the widest possible range of diversity. The use of multivalent vaccines, containing a defined mixture of immunogens from a number of prevalent HIV subtypes, might be a feasible approach to achieve broadly protective HIV vaccines.

Thus, there remains a need for improved methods and compositions for inducing immune responses against HIV. The invention provides such a method and composition. These and other advantages of the invention, as well as additional inventive features, will be apparent from the description of the invention provided herein.

### BRIEF SUMMARY OF THE INVENTION

The invention provides a method of inducing an immune response against a human immunodeficiency virus (HIV) in a mammal. The method comprises administering to the mammal an adenoviral vector composition, wherein the adenoviral vector composition comprises one or more adenoviral vectors encoding two or more different HIV antigens, whereupon the HIV antigens are produced in the mammal and an immune response against HIV is induced.

The invention also provides an adenoviral vector composition comprising (a) an adenoviral vector comprising a nucleic acid encoding an HIV clade A Env protein, (b) an adenoviral vector comprising a nucleic acid encoding an HIV clade B Env protein, (c) an adenoviral vector comprising a nucleic acid encoding an HIV clade C Env protein, and (d) an adenoviral vector comprising a nucleic acid encoding a fusion protein comprising an HIV clade B Gag protein and Pol protein.

### DETAILED DESCRIPTION OF THE INVENTION

The invention provides a method of inducing an immune response against a human immunodeficiency virus (HIV) in a mammal. The method comprises administering to the mammal an adenoviral vector composition, wherein the adenoviral vector composition comprises one or more adenoviral vectors encoding two or more different HIV antigens.

The invention also provides an adenoviral vector composition. The adenoviral vector composition comprises (a) an adenoviral vector comprising a nucleic acid encoding an HIV clade A Env protein, (b) an adenoviral vector comprising a nucleic acid encoding an HIV clade B Env protein, (c) an adenoviral vector comprising a nucleic acid encoding an HIV clade C Env protein, and (d) an adenoviral vector comprising a nucleic acid encoding a fusion protein comprising an HIV clade B Gag protein and Pol protein

An “antigen” is a molecule that triggers an immune response in a mammal. An “immune response” can entail, for example, antibody production and/or the activation of immune effector cells. An HIV antigen in the context of the invention can comprise any proteinaceous HIV molecule or portion thereof that provokes an immune response in mammal. An “HIV molecule” is a molecule that is a part of a human immunodeficiency virus, is encoded by a nucleic acid sequence of a human immunodeficiency virus, or is derived from or synthetically based upon any such molecule. Administration of an HIV antigen that provokes an immune response in accordance with the invention preferably leads to protective immunity against HIV. In this regard, an “immune response” to HIV is an immune response to any one or more HIV antigens.

Examples of suitable HIV antigens include all or part of an HIV Gag, Env, Pol, Tat, Reverse Transcriptase (RT), Vif, Vpr, Vpu, Vpo, Integrase, or Nef proteins. Preferably, each of the two or more HIV antigens comprises all or part of an HIV Gag, Env, and/or Pol protein. Suitable Env proteins are known in the art and include, for example, gp160, gp120, gp41, gp145, and gp140. In addition, an HIV antigen can be a modified Env protein that exhibits enhanced immunogenicity in vivo. For example, the antigen can be an Env protein comprising mutations in the cleavage site, fusion peptide, or interhelical coiled-coil domains of the Env protein (ΔCFI Env proteins) (see, e.g., Cao et al., J Virol., 71, 9808-9812 (1997), and Yang et al., J. Virol., 78, 4029-4036 (2004)).

Any clade of HIV is appropriate for antigen selection, including HIV clades A, B, C, D, B, MN, and the like. Thus, it will be appreciated that the following HIV antigens can be used in the inventive method: HIV clade A gp 140, Gag, Env, and/or Pol; HIV clade B gp140, Gag, Env, and/or Pol proteins; HIV clade C gp140, Gag, Env, and/or Pol proteins; and HIV clade MN gp140, Gag, Env, and/or Pol proteins. While it is preferred that the antigen is a Gag, Env, and/or Pol protein, any HIV protein or portion thereof capable of inducing an immune response in a mammal can be used in connection with the inventive method. HIV Gag, Env, and Pol proteins from the different HIV clades (e.g., HIV clades A, B, C, MN, etc.), as well as nucleic acid sequences encoding such proteins and methods for the manipulation and insertion of such nucleic acid sequences into vectors, are known (see, e.g., HIV Sequence Compendium, Division of AIDS, National Institute of Allergy and Infectious Diseases (2003), HIV Sequence Database (http://hiv-web.lanl.gov/content/hiv-db/mainpage.html), Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994)).

It will be appreciated that an entire, intact HIV protein is not required to produce an immune response. Indeed, most antigenic epitopes of HIV proteins are relatively small in size. Thus, fragments (e.g., epitopes or other antigenic fragments) of an HIV protein, such as any of the HIV proteins described herein, can be used as an HIV antigen. Antigenic fragments and epitopes of the HIV Gag, Env, and Pol proteins, as well as nucleic acid sequences encoding such antigenic fragments and epitopes, are known (see, e.g., HIV Immunology and HIV/SIV Vaccine Databases, Vol. 1, Division of AIDS, National Institute of Allergy and Infectious Diseases (2003)).

HIV antigens also include fusion proteins and polyproteins. A fusion protein can comprise one or more antigenic HIV protein fragments (e.g., epitopes) fused to one another, or fused to all or part of a different HIV protein or other polypeptide. The fusion protein can comprise all or part of any of the HIV antigens described herein. For example, all or part of an HIV Env protein (e.g., gp120 or gp 160), can be fused to all or part of the HIV Pol protein, or all or part of HIV Gag protein can be fused to all or part of the HIV Pol protein. Such fusion proteins effectively provide multiple HIV antigens in the context of the invention, and can be used to generate a more complete immune response against a given HIV pathogen as compared to that generated by a single HIV antigen. Similarly, polyproteins also can provide multiple HIV antigens. Polyproteins useful in conjunction with the invention include those that provide two or more HIV antigens, such as two or more of any of the HIV antigens described herein. Delivery of fusion proteins or polyproteins via adenoviral vector to a mammal allows exposure of an immune system to multiple antigens using a single nucleic acid sequence and, thus, conveniently allows a single composition to provide immunity against multiple HIV antigens or multiple epitopes of a single antigen. Nucleic acid sequences encoding fusion proteins and polyproteins of HIV antigens can be prepared and inserted into vectors by known methods (see, e.g., U.S. Pat. Nos. 5,130,247 and 5,130,248, Sambrook et al., supra, and Ausubel et al., supra).

The adenoviral vector composition comprises one or more adenoviral vectors encoding two or more different HIV antigens. It is understood that adenoviral vectors “encode” an antigen by way of a nucleic acid sequence that has been inserted into the adenoviral vector. HIV antigens are “different” if they comprise a different antigenic amino acid sequence. The two or more different HIV antigens can be any HIV antigens, such as two or more of the HIV antigens described herein. Preferably, the adenoviral vector composition comprises one or more adenoviral vectors encoding three or more, or even four or more different HIV antigens. It will be appreciated that exposing the immune system of a mammal to a “cocktail” of different HIV antigens can elicit a broader and more effective immune response than exposing the immune system to only a single HIV antigen.

The two or more different HIV antigens can be provided by two or more antigens from different HIV proteins (e.g., HIV Gag, Env, Pol, etc.) or different HIV clades (e.g., HIV clades A, B, C, D, E, MN, etc.). For example, an HIV Gag protein and Pol protein are different antigens. Similarly, HIV clade A Env protein and an HIV clade B Env protein are different HIV antigens. Preferably, the two or more different IV antigens comprise HIV antigens from two or more different IV clades. More preferably the adenoviral vector composition comprises adenoviral vectors encoding three or more different HIV antigens from three or more different HIV clades, or even four or more different HIV antigens from four or more different HIV clades. Alternatively, at least one of the two or more HIV antigens can be a chimeric antigen, which comprises amino acid sequences derived from the same antigen obtained from two or more different HIV clades. For example, a chimeric Env protein can comprise a portion of an Env amino acid sequence obtained from a clade A HIV and a portion of an Env amino acid sequence obtained from a clade B HIV.

The adenoviral vector composition can be provided, for example, by a composition comprising one or more adenoviral vectors (e.g., a single adenoviral vector) that each encode two or more different HIV antigens, or by a composition that comprises two or more adenoviral vectors (e.g., multiple adenoviral vectors) that each encode one or more different HIV antigens and, thereby, collectively encode two or more different HIV antigens. When the adenoviral vector composition comprises one or more adenoviral vectors (e.g., a single adenoviral vector) that each encode two or more HIV antigens, each adenoviral vector can comprise (i) a nucleic acid sequence that encodes two or more different HIV antigens (e.g., a Polyprotein or fusion protein), or (ii) two or more nucleic acid sequences that each encode a different HIV antigen. Consistent with configuration (i), it is within the scope of the invention two use an adenoviral vector comprising a nucleic acid sequence that encodes more than two different HIV antigens (e.g., three or more, four or more, or even five or more different HIV antigens) or encodes multiple copies of the same antigen, provided that it encodes at least two or more different HIV antigens. Likewise, consistent with configuration (ii), it is within the scope of the invention to use an adenoviral vector comprising several nucleic acid sequences (e.g., three or more, four or more, or even five or more different nucleic acid sequences) each encoding different HIV antigens or multiple copies of the same antigen, provided that the adenoviral vector encodes at least two different HIV antigens. Whether by configuration (i) or (ii), the adenoviral vector composition preferably comprises one or more adenoviral vectors encoding three or more, or even four or more, different HIV antigens (e.g., wherein each vector comprises a nucleic acid sequence that encodes three or more, or four or more different HIV antigens, or wherein each vector comprises three or more, or four or more nucleic acid sequences, and each nucleic acid sequence encodes a different HIV antigen). Desirably, the two or more, three or more, or four or more different HIV antigens are from two or more, three or more, or four or more different HIV clades.

Preferably, the adenoviral vector composition comprises two or more adenoviral vectors encoding the two or more different HIV antigens, and each adenoviral vector comprises a nucleic acid sequence that encodes at least one of the two or more different HIV antigens. Although the adenoviral vector composition comprises two or more adenoviral vectors encoding two or more different HIV antigens, there is no upper limit to the number of adenoviral vectors use or the number of different HIV antigens encoded thereby. Preferably, the adenoviral vector composition comprises three or more adenoviral vectors encoding three or more different HIV antigens, and each adenoviral vector comprises a nucleic acid sequence that encodes at least one of the three or more different HIV antigens. Most preferably, the adenoviral vector composition comprises four or more adenoviral vectors encoding four or more different HIV antigens, and each adenoviral vector comprises a nucleic acid sequence that encodes at least one of the four or more different HIV antigens. Desirably, the two or more, three or more, or four or more different HIV antigens are from two or more, three or mote, or four or more different HIV clades.

Of course, a combination of the above configurations of adenoviral vectors can be used without departing from the spirit and scope of the invention. For example, the adenoviral vector composition used in accordance with the invention can comprise a first adenoviral vector encoding a single HIV antigen and a second adenoviral vector encoding two or more HIV antigens that are different from the HIV antigen encoded by the first adenoviral vector. Other similar combinations and permutations of the adenoviral vector configurations disclosed herein are apparent and can be used in accordance with the invention.

When the adenoviral vector composition comprises two or more adenoviral vectors, the relative amount of each of the two or more adenoviral vectors included in the composition will depend upon a number of factors, including the immunogenicity of a particular HIV antigen compared to the other HIV antigens. The adenoviral vector composition can comprise equal amounts of each of the two or more adenoviral vectors. Alternatively, the adenoviral vector composition can comprise different amounts of each of the two or more adenoviral vectors.

In a particularly preferred embodiment of the invention, the adenoviral vector composition comprises four adenoviral vectors each comprising a nucleic acid sequence encoding a clade B Gag-Pol fusion protein, clade A gp 140, clade B gp140, and clade C gp140, respectively. Most preferably, the adenoviral vector composition comprises four adenoviral vectors having the nucleic acid sequence of SEQ ID NO: 4, SEQ ID NO: 5, SEQ ID NO: 6, and SEQ ID NO: 7. SEQ ID NO: 4 is the nucleic acid sequence of an E1/E4-deficient adenoviral vector encoding a clade B Gag-Pol fusion protein. SEQ ID NO: 5 is the nucleic acid sequence of an E1/E4-deficient adenoviral vector encoding a clade A gp140 protein. SEQ ID NO: 6 is the nucleic acid sequence of an E1/E4-deficient adenoviral vector encoding a clade B gp140 protein. SEQ ID NO: 7 is the nucleic acid sequence of a clade C gp140 protein. Desirably, the adenoviral vector composition comprises the following adenoviral vectors in a 3:1:1:1 ratio by weight, respectively: an adenoviral vector comprising a nucleic acid sequence encoding clade B Gag-Pol fusion protein, an adenoviral vector comprising a nucleic acid sequence encoding clade A gp140, an adenoviral vector comprising a nucleic acid sequence encoding clade B gp 140, and an adenoviral vector comprising a nucleic acid sequence encoding clade C gp140.

Typically, the adenoviral vector comprises a nucleic acid encoding one or more HIV antigens as part of an expression cassette, i.e., a particular nucleotide sequence that possesses functions which facilitate subcloning and recovery of a nucleic acid sequence (e.g., one or more restriction sites) or expression of a nucleic acid sequence (e.g., Polyadenylation or splice sites). The nucleic acid preferably is located in the E1 region (e.g., replaces the E1 region in whole or in part) or the E4 region of the adenoviral genome. For example, the E1 region can be replaced by a promoter-variable expression cassette comprising a nucleic acid encoding an antigen. The expression cassette optionally can be inserted in a 3 ′-5′ orientation, e.g., oriented such that the direction of transcription of the expression cassette is opposite that of the surrounding adjacent adenoviral genome. However, it is also appropriate for the expression cassette to be inserted in a 5 ′-3′ orientation with respect to the direction of transcription of the surrounding genome. In addition to the expression cassette comprising the nucleic acid encoding an antigen, the adenoviral vector can comprise other expression cassettes containing other exogenous nucleic acids, which cassettes can replace any of the deleted regions of the adenoviral genome. The insertion of an expression cassette into the adenoviral genome (e.g., into the E1 region of the genome) can be facilitated by known methods, for example, by the introduction of a unique restriction site at a given position of the adenoviral genome. As set forth above, preferably all or part of the E3 region of the adenoviral vector also is deleted.

Preferably, the antigen-encoding nucleic acid is operably linked to (i.e., under the transcriptional control of) one or more promoter and/or enhancer elements, for example, as part of a promoter-variable expression cassette. Techniques for operably linking sequences together are well known in the art. A “promoter” is a DNA sequence that directs the binding of RNA Polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the invention to provide for transcription of the nucleic acid sequence. The promoter preferably is capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

Promoter regions can vary in length and sequence and can further encompass one or more DNA binding sites for sequence-specific DNA binding proteins and/or an enhancer or silencer. Enhancers and/or silencers can similarly be present on a nucleic acid sequence outside of the promoter per se. Desirably, a cellular or viral enhancer, such as the cytomegalovirus (CMV) immediate-early enhancer, is positioned in the proximity of the promoter to enhance promoter activity. In addition, splice acceptor and donor sites can be present on a nucleic acid sequence to enhance transcription.

Any suitable promoter or enhancer sequence can be used in the context of the invention. In this respect, the antigen-encoding nucleic acid sequence can be operably linked to a viral promoter. Suitable viral promoters include, for instance, cytomegalovirus (CMV) promoters, such as the CMV immediate-early promoter (described in, for example, U.S. Pat. Nos. 5,168,062 and 5,385,839), promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, and the like.

Alternatively, the invention employs a cellular promoter, i.e., a promoter that drives expression of a cellular protein. Preferred cellular promoters for use in the invention will depend on the desired expression profile to produce the antigen(s). In one aspect, the cellular promoter is preferably a constitutive promoter that works in a variety of cell types, such as immune cells described herein. Suitable constitutive promoters can drive expression of genes encoding transcription factors, housekeeping genes, or structural genes common to eukaryotic cells. For example, the Ying Yang 1 (YY1) transcription factor (also referred to as NMP-1, NF-E1, and UCRBP) is a ubiquitous nuclear transcription factor that is an intrinsic component of the nuclear matrix (Guo et al., PNAS, 92, 10526-10530 (1995)). While the promoters described herein are considered constitutive promoters, it is understood in the art that constitutive promoters can be upregulated. Promoter analysis shows that the elements critical for basal transcription reside from −277 to +475 of the YY1 gene relative to the transcription start site from the promoter, and include a TATA and CCAAT box. JEM-1 (also known as HGMW and BLZF-1) also is a ubiquitous nuclear transcription factor identified in normal and tumorous tissues (Tong et al., Leukemia, 12(11), 1733-1740 (1998), and Tong et al., Genomics, 69(3), 380-390 (2000)). JEM-1 is involved in cellular growth control and maturation, and can be upregulated by retinoic acids. Sequences responsible for maximal activity of the JEM-1 promoter has been located at −432 to +101 of the JEM-1 gene relative the transcription start site of the promoter. Unlike the YY1 promoter, the JEM-1 promoter does not comprise a TATA box. The ubiquitin promoter, specifically UbC, is a strong constitutively active promoter functional in several species. The UbC promoter is further characterized in Marinovic et al., J. Biol. Chem., 277(19), 16673-16681 (2002).

Many of the above-described promoters are constitutive promoters. Instead of being a constitutive promoter, the promoter can be a regulatable promoter, i.e., a promoter that is up- and/or down-regulated in response to appropriate signals. The use of a regulatable promoter or expression control sequence is particularly applicable to DNA vaccine development as antigenic proteins, including viral and parasite antigens, frequently are toxic to complementing cell lines. In one embodiment, the regulatory sequences operably linked to the antigen-encoding nucleic acid sequence include components of the tetracycline expression system, e.g., the operator sites. For instance, the antigen-encoding nucleic acid sequence is operably linked to a promoter which is operably linked to one or more tet operator sites. An adenoviral vector comprising such an expression cassette can be propagated in a complementing cell line, such as 293-ORF6 described in, for example, U.S. Pat. No. 5,994,106 and International Patent Application Publication WO 95/34671, which comprises a nucleic acid sequence encoding a tet repressor protein. By producing the tet repressor protein in the complementing cell line, antigen production is inhibited and propagation proceeds without any associated antigen-mediated toxicity. Suitable regulatable promoter systems also include, but are not limited to, the IL-8 promoter; the metallothionine inducible promoter system, the bacterial lacZYA expression system, and the T7 Polymerase system. Further, promoters that are selectively activated at different developmental stages (e.g., globin genes are differentially transcribed from globin-associated promoters in embryos and adults) can be employed. The promoter sequence can contain at least one regulatory sequence responsive to regulation by an exogenous agent. The regulatory sequences are preferably responsive to exogenous agents such as, but not limited to, drugs, hormones, radiation, or other gene products.

The promoter can be a tissue-specific promoter, i.e., a promoter that is preferentially activated in a given tissue and results in expression of a gene product in the tissue where activated. A tissue-specific promoter suitable for use in the invention can be chosen by the ordinarily skilled artisan based upon the target tissue or cell-type. Preferred tissue-specific promoters for use in the inventive method are specific to immune cells, such as the dendritic-cell specific Dectin-2 promoter described in Morita et al., Gene Ther., 8, 1729-37 (2001).

In yet another embodiment, the promoter can be a chimeric promoter. A promoter is “chimeric” in that it comprises at least two nucleic acid sequence portions obtained from, derived from, or based upon at least two different sources (e.g., two different regions of an organism's genome, two different organisms, or an organism combined with a synthetic sequence). Preferably, the two different nucleic acid sequence portions exhibit less than about 40%, more preferably less than about 25%, and even more preferably less than about 10% nucleic acid sequence identity to one another (which can be determined by methods described elsewhere herein). Any suitable chimeric promoter can be used in the inventive method.

A promoter can be selected for use in the invention by matching its particular pattern of activity with the desired pattern and level of expression of the antigen(s). For example, in embodiments where the adenoviral vector comprises two or more nucleic acid sequences that encode different antigens, each nucleic acid sequence can be operably linked to different promoters displaying distinct expression profiles. For example, a first promoter is selected to mediate an initial peak of antigen production, thereby priming the immune system against an encoded antigen. A second promoter is selected to drive production of the same or different antigen such that expression peaks several days after that of the first promoter, thereby “boosting” the immune system against the antigen. Alternatively, a chimeric promoter can be constructed which combines the desirable aspects of multiple promoters. For example, a CMV-RSV hybrid promoter combining the CMV promoter's initial rush of activity with the RSV promoter's high maintenance level of activity is especially preferred for use in many embodiments of the inventive method. In that antigens can be toxic to eukaryotic cells, it may be advantageous to modify the promoter to decrease activity in complementing cell lines used to propagate the adenoviral vector.

To optimize protein production, preferably the antigen-encoding nucleic acid sequence further comprises a polyadenylation site following the coding sequence of the antigen-encoding nucleic acid sequence. Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (Human Sarcoma Virus-40) polyadenylation sequence. Also, preferably all the proper transcription signals (and translation signals, where appropriate) are correctly arranged such that the nucleic acid sequence is properly expressed in the cells into which it is introduced. If desired, the nucleic acid sequence also can incorporate splice sites (i.e., splice acceptor and splice donor sites) to facilitate MnRNA production.

If the antigen-encoding nucleic acid sequence encodes a processed or secreted protein or peptide, or a protein that acts intracellularly, preferably the antigen-encoding nucleic acid sequence further comprises the appropriate sequences for processing, secretion, intracellular localization, and the like. The antigen-encoding nucleic acid sequence can be operably linked to a signal sequence, which targets a protein to cellular machinery for secretion. Appropriate signal sequences include, but are not limited to, leader sequences for immunoglobulin heavy chains and cytokines, (see, for example, Ladunga, Current Opinions in Biotechnology, 11, 13-18 (2000)). Other protein modifications can be required to secrete a protein from a host cell, which can be determined using routine laboratory techniques. Preparing expression constructs encoding antigens and signal sequences is further described in, for example, U.S. Pat. No. 6,500,641. Methods of secreting non-secretable proteins are further described in, for example, U.S. Pat. No. 6,472,176, and International Patent Application Publication WO 02/48377.

An antigen protein encoded by the nucleic acid sequence of the adenoviral vector also can be modified to attach or incorporate the antigen on the host cell surface. In this respect, the antigen can comprise a membrane anchor, such as a gpi-anchor, for conjugation onto the cell surface. A transmembrane domain can be fused to the antigen to incorporate a terminus of the antigen protein into the cell membrane. Other strategies for displaying peptides on a cell surface are known in the art and are appropriate for use in the context of the invention.

In accordance with the invention, the adenoviral vector composition is administered to an animal, preferably a mammal (e.g., a human), wherein each antigen-encoding nucleic acid sequence is expressed to induce an immune response against the antigen. The immune response can be a humoral immune response, a cell-mediated immune response, or, desirably, a combination of humoral and cell-mediated immunity. Ideally, the immune response provides protection upon subsequent challenge with the infectious agent comprising the antigen. However, protective immunity is not required in the context of the invention. The inventive method further can be used for antibody production and harvesting.

To enhance the immune response generated against an HIV antigen, the adenoviral vector composition can also comprise a nucleic acid sequence that encodes an immune stimulator, such as a cytokine, a chemokine, or a chaperone. Cytokines include, for example, Macrophage Colony Stimulating Factor (e.g., GM-CSF), Interferon Alpha (IFN-α), Interferon Beta (IFN-β), Interferon Gamma (IFN-γ), interleukins (IL 1, IL-2, IL-4, IL-5, IL-6, IL-8, IL-10, IL-12, IL-13, IL-15, IL-16, and IL-18), the TNF family of proteins, Intercellular Adhesion Molecule-1 (ICAM-1), Lymphocyte Function-Associated antigen-3 (LFA-3), B7-1, B7-2, FMS-related tyrosine kinase 3 ligand, (Flt3L), vasoactive intestinal peptide (VIP), and CD40 ligand. Chemolcines include, for example, B Cell-Attracting chemokine-I (BCA-1), Fractalkine, Melanoma Growth Stimulatory Activity protein (MGSA), Hemofiltrate CC chemokine 1 (HCC-1), Interleukin 8 (IL8), Interferon-stimulated T-cell alpha chemoattractant (I-TAC), Lymphotactin, Monocyte Chemotactic Protein 1 (MCP-1), Monocyte Chemotactic Protein 3 (MCP-3), Monocyte Chemotactic Protein 4 (MCP-4), Macrophage-Derived Chemokine (MDC), a macrophage inflammatory protein (MIP), Platelet Factor 4 (PF4), RANTES, BRAK, eotaxin, exodus 1-3, and the like. Chaperones include, for example, the heat shock proteins Hsp 170, Hsp70, and Hsp40. Cytokines and chemokines are generally described in the art, including the Invivogen catalog (2002), San Diego, Calif.

Administration of the adenoviral vector composition can be one component of a multistep regimen for inducing an immune response against HIV in a mammal. In this respect, the inventive method further comprises administering to the mammal a primer composition comprising one or more nucleic acid sequences that encode at least one HIV antigen that is the same as an HIV antigen encoded by an adenoviral vector of the adenoviral vector composition, wherein the administration of the primer composition is performed at least one week before the administration of the adenoviral vector composition. Thus, this embodiment of the invention represents one arm of a prime and boost immunization regimen, wherein an immune response is “primed” by administration of the primer composition, and is “boosted” by administration of the adenoviral vector composition. The one or more nucleic acid sequences of the primer composition can be administered as part of a gene transfer vector or as naked DNA. Any gene transfer vector can be employed in the primer composition, including viral and non-viral gene transfer vectors. Examples of suitable viral gene transfer vectors include, but are not limited to, retroviral vectors, adeno-associated virus vectors, vaccinia virus vectors, herpesvirus vectors, or adenoviral vectors. Examples of suitable non-viral vectors include, but are not limited to, plasmids, liposomes, and molecular conjugates (e.g., transferrin). Ideally, the gene transfer vector is a plasmid or an adenoviral vector. Alternatively, an immune response can be primed or boosted by administration of the antigen itself, e.g., an antigenic protein, inactivated pathogen, and the like.

While the antigen encoded by the one or more nucleic acid sequences of the primer composition preferably is the same as an HIV antigen encoded by an adenoviral vector of the adenoviral vector composition, in some embodiments it may be appropriate to use a primer composition comprising one or more nucleic acid sequences encoding an HIV antigen that is different from the antigen(s) encoded by the adenoviral vector composition. Preferably, the primer composition comprises one or more nucleic acid sequences that encode two or more HIV antigens that are the same as the HIV antigens encoded by the one or more adenoviral vectors of the adenoviral vector composition. More preferably, the primer composition comprises one or more nucleic acid sequences that encode all of the HIV antigens encoded by the one or more adenoviral vectors of the adenoviral vector composition.

The primer composition is administered to the mammal to prime the immune response to HIV. More than one dose of primer composition can be provided in any suitable timeframe (e.g., at least about 1 week, 2 weeks, 4 weeks, 8 weeks, 12 weeks, 16 weeks, or more prior to boosting). Preferably, the primer composition is administered to the mammal at least three months (e.g., three, six, nine, twelve, or more months) before administration of the adenoviral vector composition. Most preferably, the primer composition is administered to the mammal at least about six months to about nine months before administration of the adenoviral vector composition. The adenoviral vector composition is administered to the mammal to boost the immune response to HIV. More than one dose of adenoviral vector composition can be provided in any suitable timeframe to maintain immunity.

The adenoviral vector composition and/or the primer composition desirably is administered in a pharmaceutically acceptable (e.g., physiologically acceptable) composition, which comprises a carrier, preferably a physiologically (e.g., pharmaceutically) acceptable carrier and the adenoviral vector composition. Any suitable carrier can be used within the context of the invention, and such carriers are well known in the art. The choice of carrier will be determined, in part, by the particular site to which the composition is to be administered and the particular method used to administer the composition. Ideally, in the context of adenoviral vectors, the pharmaceutical composition preferably is free of replication-competent adenovirus. The pharmaceutical composition can optionally be sterile or sterile with the exception of the one or more adenoviral vectors.

Suitable formulations for the pharmaceutical composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the pharmaceutical composition for use in the inventive method is formulated to protect the adenoviral vectors from damage prior to administration. For example, the pharmaceutical composition can be formulated to reduce loss of the adenoviral vectors on devices used to prepare, store, or administer the expression vector, such as glassware, syringes, or needles. The pharmaceutical composition can be formulated to decrease the light sensitivity and/or temperature sensitivity of the adenoviral vectors. To this end, the pharmaceutical composition preferably comprises a pharmaceutically acceptable liquid carrier, such as, for example, those described above, and a stabilizing agent selected from the group consisting of Polysorbate 80, L-arginine, polyvinylpyrrolidone, trehalose, and combinations thereof. Use of such a composition will extend the shelf life of the vector, facilitate administration, and increase the efficiency of the inventive method. Formulations for adenoviral vector-containing compositions are further described in, for example, U.S. Pat. Nos. 6,225,289, 6,514,943, U.S. Patent Application Publication No. 2003/0153065 A1, and International Patent Application Publication WO 00/34444. A pharmaceutical composition also can be formulated to enhance transduction efficiency of the adenoviral vector. In addition, one of ordinary skill in the art will appreciate that the pharmaceutical composition can comprise other therapeutic or biologically-active agents. For example, factors that control inflammation, such as ibuprofen or steroids, can be part of the pharmaceutical composition to reduce swelling and inflammation associated with in vivo administration of the adenoviral vectors. As discussed herein, immune system stimulators can be administered to enhance any immune response to the antigens. Antibiotics, i.e., microbicides and fungicides, can be present to treat existing infection and/or reduce the risk of future infection, such as infection associated with gene transfer procedures.

Any route of administration can be used to deliver the pharmaceutical composition to the mammal. Indeed, although more than one route can be used to administer the pharmaceutical composition, a particular route can provide a more immediate and more effective reaction than another route. Preferably, the pharmaceutical composition is administered via intramuscular injection. The pharmaceutical composition also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The pharmaceutical composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the pharmaceutical composition. The pharmaceutical composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the pharmaceutical composition administered to the mammal will depend on a number of factors, including the size of a target tissue, the extent of any side-effects, the particular route of administration, and the like. The dose ideally comprises an “effective amount” of adenoviral vector composition and/or the primer composition, i.e., a dose of adenoviral vector composition and/or the primer composition which provokes a desired immune response in the mammal. The desired immune response can entail production of antibodies, protection upon subsequent challenge, immune tolerance, immune cell activation, and the like. In embodiments where the adenoviral vector composition comprises two or more adenoviral vectors, it will be appreciated that the pharmaceutical composition of the inventive method comprises a dose of adenoviral vector that is the combined dose of each of the two or more adenoviral vectors contained therein.

Desirably, the adenoviral vector composition comprises a single dose of adenoviral vector comprising at least about 1×10⁵ particles (which also is referred to as particle units) of adenoviral vector. The dose preferably is at least about 1×10⁶ particles (e.g., about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ particles). In other words, the adenoviral vector composition can comprise a single dose of adenoviral vector comprising, for example, about 1×10⁶ particle units (pu), 2×10⁶ pu, 4×10⁶ pu, 1×10⁷ pu, 2×10⁷ pu, 4×10⁷ pu, 1×10⁸ pu, 2×10⁸ pu, 4×10⁸ pu, 1×10⁹ pu, 2×10⁹ pu, 4×10⁹ pu, 1×10¹⁰ pu, 2×10¹⁰ pu, 4×10¹⁰ pu, 1×10¹¹ pu, 2×10¹¹ pu, 4×10¹¹ pu, 1×10¹² pu, 2×10¹² Pu, or 4×10¹² pu of adenoviral vector.

The primer composition desirably comprises at least about 1 mg of nucleic acid, typically and preferably DNA. The primer composition preferably comprises 1 mg or more of nucleic acid (e.g., about 1 mg, 2 mg, 3 mg, 4 mg, 5 mg, or more). In a preferred embodiment, the primer composition comprises about 2 mg to about 5 mg nucleic acid (e.g., about 3 mg or 4 mg), more preferably about 3 mg to about 5 mg nucleic acid (e.g., about 3.5 mg), and most preferably about 4 mg to about 5 mg nucleic acid (e.g., about 4.5 mg).

Modified viruses have proven convenient vector systems for investigative and therapeutic gene transfer applications, and adenoviral vector systems present several advantages for such uses. Adenoviruses are generally associated with benign pathologies in humans, and the 36 kilobase (kb) adenoviral genome has been extensively studied. Adenoviral vectors can be produced in high titers (e.g., about 10¹³ particle forming units (pfu)), and such vectors can transfer genetic material to nonreplicating, as well as replicating, cells; in contrast with, e.g., retroviral vectors, which only transfer genetic material to replicating cells. The adenoviral genome can be manipulated to carry a large amount of exogenous DNA (up to about 8 kb), and the adenoviral capsid can potentiate the transfer of even longer sequences (Curiel et al., Hum. Gene Ther., 3, 147-154 (1992)). Additionally, adenoviruses generally do not integrate into the host cell chromosome, but rather are maintained as a linear episome, thus minimizing the likelihood that a recombinant adenovirus will interfere with normal cell function. In addition to being a superior vehicle for transferring genetic material to a wide variety of cell types, adenoviral vectors represent a safe choice for gene transfer, a particular concern for therapeutic applications.

Adenovirus from various origins, subtypes, or mixture of subtypes can be used as the source of the viral genome for the adenoviral vector. While non-human adenovirus (e.g., simian, avian, canine, ovine, or bovine adenoviruses) can be used to generate the adenoviral vector, a human adenovirus preferably is used as the source of the viral genome for the adenoviral vector of the inventive method. Adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Preferably, in the context of the inventive method, the adenoviral vector is of human subgroup C, especially serotype 2 or even more desirably serotype 5. However, non-group C adenoviruses can be used to prepare adenoviral gene transfer vectors for delivery of gene products to host cells. Preferred adenoviruses used in the construction of non-group C adenoviral gene transfer vectors include Ad12 (group A), Ad7 and Ad35 (group B), Ad30 and Ad36 (group D), Ad4 (group E), and Ad4l (group F). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561 and International Patent Applications WO 97/12986 and WO 98/53087.

The adenoviral vector can comprise a mixture of subtypes and thereby be a “chimeric” adenoviral vector. A chimeric adenoviral vector can comprise an adenoviral genome that is derived from two or more (e.g., 2, 3, 4, etc.) different adenovirus serotypes. In the context of the invention, a chimeric adenoviral vector can comprise approximately equal amounts of the genome of each of the two or more different adenovirus serotypes. When the chimeric adenoviral vector genome is comprised of the genomes of two different adenovirus serotypes, the chimeric adenoviral vector genome preferably comprises no more than about 70% (e.g., no more than about 65%, about 50%, or about 40%) of the genome of one of the adenovirus serotypes, with the remainder of the chimeric adenovirus genome being derived from the genome of the other adenovirus serotype. In one embodiment, the chimeric adenoviral vector can contain an adenoviral genome comprising a portion of a serotype 2 genome and a portion of a serotype 5 genome. For example, the 5′ region of an adenoviral serotype 5 genome (i.e., the region of the genome 5′ to the adenoviral E1 region) can be replaced with the corresponding region of an adenoviral serotype 2 genome (e.g., the Ad5 genome region 5′ to the E1 region of the adenoviral genome is replaced with nucleotides 1-456 of the Ad2 genome).

The adenoviral vector of the invention can be replication-competent. For example, the adenoviral vector can have a mutation (e.g., a deletion, an insertion, or a substitution) in the adenoviral genome that does not inhibit viral replication in host cells. The inventive adenoviral vector also can be conditionally replication-competent. Preferably, however, the adenoviral vector is replication-deficient in host cells.

By “replication-deficient” is meant that the adenoviral vector requires complementation of one or more regions of the adenoviral genome that are required for replication, as a result of, for example a deficiency in at least one replication-essential gene function (i.e., such that the adenoviral vector does not replicate in typical host cells, especially those in a human patient that could be infected by the adenoviral vector in the course of the inventive method). A deficiency in a gene, gene function, or genomic region, as used herein, is defined as a deletion of sufficient genetic material of the viral genome to obliterate or impair the function of the gene (e.g., such that the function of the gene product is reduced by at least about 2-fold, 5-fold, 10-fold, 20-fold, 30-fold, or 50-fold) whose nucleic acid sequence was deleted in whole or in part. Deletion of an entire gene region often is not required for disruption of a replication-essential gene function. However, for the purpose of providing sufficient space in the adenoviral genome for one or more transgenes, removal of a majority of a gene region may be desirable. While deletion of genetic material is preferred, mutation of genetic material by addition or substitution also is appropriate for disrupting gene function. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA1 and/or VA-RNA-2).

The replication-deficient adenoviral vector desirably requires complementation of at least one replication-essential gene function of one or more regions of the adenoviral genome. Preferably, the adenoviral vector requires complementation of at least one gene function of the E1A region, the E1B region, or the E4 region of the adenoviral genome required for viral replication (denoted an E1-deficient or E4-deficient adenoviral vector). In addition to a deficiency in the E1 region, the recombinant adenovirus also can have a mutation in the major late promoter (MLP), as discussed in International Patent Application Publication WO 00/00628. Most preferably, the adenoviral vector is deficient in at least one replication-essential gene function (desirably all replication-essential gene functions) of the E1 region and at least one gene function of the nonessential E3 region (e.g., an Xba I deletion of the E3 region) (denoted an E1/E3-deficient adenoviral vector). With respect to the E1 region, the adenoviral vector can be deficient in part or all of the E1 A region and/or part or all of the E1B region, e.g., in at least one replication-essential gene function of each of the E1A and E1B regions, thus requiring complementation of the E1A region and the E1B region of the adenoviral genome for replication. The adenoviral vector also can require complementation of the E4 region of the adenoviral genome for replication, such as through a deficiency in one or more replication-essential gene functions of the E4 region.

When the adenoviral vector is E1-deficient, the adenoviral vector genome can comprise a deletion beginning at any nucleotide between nucleotides 335 to 375 (e.g., nucleotide 356) and ending at any nucleotide between nucleotides 3,310 to 3,350 (e.g., nucleotide 3,329) or even ending at any nucleotide between 3,490 and 3,530 (e.g., nucleotide 3,510) (based on the adenovirus serotype 5 genome).

When E2A-deficient, the adenoviral vector genome can comprise a deletion beginning at any nucleotide between nucleotides 22,425 to 22,465 (e.g., nucleotide 22,443) and ending at any nucleotide between nucleotides 24,010 to 24,050 (e.g., nucleotide 24,032) (based on the adenovirus serotype 5 genome). When E3-deficient, the adenoviral vector genome can comprise a deletion beginning at any nucleotide between nucleotides 28,575 to 29,615 (e.g., nucleotide 28,593) and ending at any nucleotide between nucleotides 30,450 to 30,490 (e.g., nucleotide 30,470) (based on the adenovirus serotype 5 genome).

When the adenoviral vector is deficient in at least one replication-essential gene function in one region of the adenoviral genome (e.g., an E1- or E1/E3-deficient adenoviral vector), the adenoviral vector is referred to as “singly replication-deficient.” A particularly preferred singly replication-deficient adenoviral vector is, for example, a replication-deficient adenoviral vector requiring, at most, complementation of the E1 region of the adenoviral genome, so as to propagate the adenoviral vector (e.g., to form adenoviral vector particles).

The adenoviral vector of the invention can be “multiply replication-deficient,” meaning that the adenoviral vector is deficient in one or more replication-essential gene functions in each of two or more regions of the adenoviral genome, and requires complementation of those functions for replication. For example, the aforementioned E1-deficient or E1/E3-deficient adenoviral vector can be further deficient in at least one replication-essential gene function of the E4 region (denoted an E1/E4- or E1/E3/E4-deficient adenoviral vector), and/or the E2 region (denoted an El/E2- or E1/E2/E3-deficient adenoviral vector), preferably the E2A region (denoted an E1/E2A- or E1/E2A/E3-deficient adenoviral vector). An adenoviral vector deleted of the entire E4 region can elicit a lower host immune response. When E4-deficient, the adenoviral vector genome can comprise a deletion beginning at, for example, any nucleotide between nucleotides 32,805 to 32,845 (e.g., nucleotide 32,826) and ending at, for example, any nucleotide between nucleotides 35,540 to 35,580 (e.g., nucleotide 35,561) (based on the adenovirus serotype 5 genome), optionally in addition to deletions in the E1 region (e.g., nucleotides 356 to 3,329 or nucleotides 356 to 3,510) (based on the adenovirus serotype 5 genome) and/or deletions in the E3 region (e.g., nucleotides 28,594 to 30,469 or nucleotides 28,593 to 30,470) (based on the adenovirus serotype 5 genome). The endpoints defining the deleted nucleotide portions can be difficult to precisely determine and typically will not significantly affect the nature of the adenoviral vector, i.e., each of the aforementioned nucleotide numbers can be ±1, 2, 3, 4, 5, or even 10 or 20 nucleotides.

If the adenoviral vector of the invention is deficient in a replication-essential gene function of the E2A region, the vector preferably does not comprise a complete deletion of the E2A region, which deletion preferably is less than about 230 base pairs in length. Generally, the E2A region of the adenovirus codes for a DBP (DNA binding protein), a Polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (13rough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. It is preferable that any multiply replication-deficient adenoviral vector contains this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5. This portion of the adenoviral genome desirably is included in the adenoviral vector because it is not complemented in current E2A cell lines so as to provide the desired level of viral propagation.

While the above-described deletions are described with respect to an adenovirus serotype 5 genome, one of ordinary skill in the art can determine the nucleotide coordinates of the same regions of other adenovirus serotypes, such as an adenovirus serotype 2 genome, without undue experimentation, based on the similarity between the genomes of various adenovirus serotypes, particularly adenovirus serotypes 2 and 5.

In one embodiment of the inventive method, the adenoviral vector can comprise an adenoviral genome deficient in one or more replication-essential gene functions of each of the E1 and E4 regions (i.e., the adenoviral vector is an E1/E4-deficient adenoviral vector), preferably with the entire coding region of the E4 region having been deleted from the adenoviral genome. In other words, all the open reading frames (ORFs) of the E4 region have been removed. Most preferably, the adenoviral vector is rendered replication-deficient by deletion of all of the E1 region and by deletion of a portion of the E4 region. The E4 region of the adenoviral vector can retain the native E4 promoter, Polyadenylation sequence, and/or the right-side inverted terminal repeat (ITR).

It should be appreciated that the deletion of different regions of the adenoviral vector can alter the immune response of the mammal. In particular, deletion of different regions can reduce the inflammatory response generated by the adenoviral vector. Furthermore, the adenoviral vector's coat protein can be modified so as to decrease the adenoviral vector's ability or inability to be recognized by a neutralizing antibody directed against the wild-type coat protein, as described in International Patent Application WO 98/40509. Such modifications are useful for long-term treatment of persistent ocular disorders.

The adenoviral vector, when multiply replication-deficient, especially in replication-essential gene functions of the E1 and E4 regions, can include a spacer sequence to provide viral growth in a complementing cell line similar to that achieved by singly replication-deficient adenoviral vectors, particularly an E1-deficient adenoviral vector. In a preferred E4-deficient adenoviral vector of the invention wherein the L5 fiber region is retained, the spacer is desirably located between the L5 fiber region and the right-side ITR. More preferably in such an adenoviral vector, the E4 Polyadenylation sequence alone or, most preferably, in combination with another sequence exists between the L5 fiber region and the right-side ITR, so as to sufficiently separate the retained L5 fiber region from the right-side ITR, such that viral production of such a vector approaches that of a singly replication-deficient adenoviral vector, particularly a singly replication-deficient E1 deficient adenoviral vector.

The spacer sequence can contain any nucleotide sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer sequence can be coding or non-coding and native or non-native with respect to the adenoviral genome, but does not restore the replication-essential function to the deficient region. The spacer can also contain a promoter-variable expression cassette. More preferably, the spacer comprises an additional Polyadenylation sequence and/or a passenger gene. Preferably, in the case of a spacer inserted into a region deficient for E4, both the E4 Polyadenylation sequence and the E4 promoter of the adenoviral genome or any other (cellular or viral) promoter remain in the vector. The spacer is located between the E4 Polyadenylation site and the E4 promoter, or, if the E4 promoter is not present in the vector, the spacer is proximal to the right-side ITR. The spacer can comprise any suitable Polyadenylation sequence. Examples of suitable Polyadenylation sequences include synthetic optimized sequences, BGH (Bovine Growth Hormone), Polyoma virus, TK (Thymidine Kinase), EBV (Epstein Barr Virus) and the papillomaviruses, including human papillomaviruses and BPV (Bovine Papilloma Virus). Preferably, particularly in the E4 deficient region, the spacer includes an SV40 Polyadenylation sequence. The SV40 Polyadenylation sequence allows for higher virus production levels of multiply replication deficient adenoviral vectors. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. Ideally, the spacer comprises the glucuronidase gene. The use of a spacer in an adenoviral vector is further described in, for example, U.S. Pat. No. 5,851,806 and International Patent Application WO 97/21826.

It has been observed that an at least E4-deficient adenoviral vector expresses a transgene at high levels for a limited amount of time in vivo and that persistence of expression of a transgene in an at least E4-deficient adenoviral vector can be modulated through the action of a trans-acting factor, such as HSV ICP0, Ad pTP, CMV-IE2, CMV-IE86, HIV tat, HTLV-tax, HBV-X, AAV Rep 78, the cellular factor from the U205 osteosarcoma cell line that functions like HSV ICP0, or the cellular factor in PC12 cells that is induced by nerve growth factor, among others, as described in for example, U.S. Pat. Nos. 6,225,113, 6,649,373, and 6,660,521, and International Patent Application Publication WO 00/34496. In view of the above, a multiply deficient adenoviral vector (e.g., the at least E4 deficient adenoviral vector) or a second expression vector can comprise a nucleic acid sequence encoding a trans-acting factor that modulates the persistence of expression of the nucleic acid sequence. Persistent expression of antigenic DNA can be desired when generating immune tolerance.

Desirably, the adenoviral vector requires, at most, complementation of replication-essential gene functions of the E1, E2A, and/or E4 regions of the adenoviral genome for replication (i.e., propagation). However, the adenoviral genome can be modified to disrupt one or more replication-essential gene functions as desired by the practitioner, so long as the adenoviral vector remains deficient and can be propagated using, for example, complementing cells and/or exogenous DNA (e.g., helper adenovirus) encoding the disrupted replication-essential gene functions. In this respect, the adenoviral vector can be deficient in replication-essential gene functions of only the early regions of the adenoviral genome, only the late regions of the adenoviral genome, and both the early and late regions of the adenoviral genome. An adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). Suitable replication-deficient adenoviral vectors, including multiply replication-deficient adenoviral vectors, are disclosed in U.S. Pat. Nos. 5,837,511; 5,851,806; 5,994,106; 6,127,175; and 6,482,616; U.S. Patent Application Publications 2001/0043922 A1, 2002/0004040 A1, 2002/0031831 A1, 2002/0110545 A1, and 2004/0161848 A1, and International Patent Application Publications WO 94/28152, WO 95/02697, WO 95/16772, WO 95/34671, WO 96/22378, WO 97/12986, WO 97/21826, and WO 03/022311.

Ideally, the adenoviral vector administered to a mammal is in the form of an adenoviral vector composition, especially a pharmaceutical composition, which is virtually free of replication-competent adenovirus (RCA) contamination (e.g., the composition comprises less than about 1% of RCA contamination). Most desirably, the composition is RCA-free. Adenoviral vector compositions and stocks that are RCA-free are described in U.S. Pat. Nos. 5,944,106 and 6,482,616, U.S. Published Patent Application 2002/0110545 A1, and International Patent Application WO 95/34671.

By removing all or part of, for example, the E1, E3, and E4 regions of the adenoviral genome, the resulting adenoviral vector is able to accept inserts of exogenous nucleic acid sequences while retaining the ability to be packaged into adenoviral capsids. The nucleic acid sequence can be positioned in the E1 region, the E3 region, or the E4 region of the adenoviral genome. Indeed, the nucleic acid sequence can be inserted anywhere in the adenoviral genome so long as the position does not prevent expression of the nucleic acid sequence or interfere with packaging of the adenoviral vector.

If the adenoviral vector is not replication-deficient, ideally the adenoviral vector is manipulated to limit replication of the vector to within a target tissue. The adenoviral vector can be a conditionally-replicating adenoviral vector, which is engineered to replicate under conditions pre-determined by the practitioner. For example, replication-essential gene functions, e.g., gene functions encoded by the adenoviral early regions, can be operably linked to an inducible, repressible, or tissue-specific transcription control sequence, e.g., promoter. In this embodiment, replication requires the presence or absence of specific factors that interact with the transcription control sequence. In autoimmune disease treatment, it can be advantageous to control adenoviral vector replication in, for instance, lymph nodes, to obtain continual antigen production and control immune cell production. Conditionally-replicating adenoviral vectors are described further in U.S. Pat. No. 5,998,205.

In addition to modification (e.g., deletion, mutation, or replacement) of adenoviral sequences encoding replication-essential gene functions, the adenoviral genome can contain benign or non-lethal modifications, i.e., modifications which do not render the adenovirus replication-deficient, or, desirably, do not adversely affect viral functioning and/or production of viral proteins, even if such modifications are in regions of the adenoviral genome that otherwise contain replication-essential gene functions. Such modifications commonly result from DNA manipulation or serve to facilitate expression vector construction. For example, it can be advantageous to remove or introduce restriction enzyme sites in the adenoviral genome. Such benign mutations often have no detectable adverse effect on viral functioning. For example, the adenoviral vector can comprise a deletion of nucleotides 10,594 and 10,595 (based on the adenoviral serotype 5 genome), which are associated with VA-RNA-1 transcription, but the deletion of which does not prohibit production of VA-RNA-1. (00721 Similarly, the coat protein of an adenoviral vector can be manipulated to alter the binding specificity or recognition of the adenovirus for a viral receptor on a potential host cell. For adenovirus, such manipulations can include deletion of regions of the fiber, penton, or hexon, insertions of various native or non-native ligands into portions of the coat protein, and the like. Manipulation of the coat protein can broaden the range of cells infected by an adenoviral vector or enable targeting of an adenoviral vector to a specific cell type.

For example, in one embodiment, the adenoviral vector comprises a chimeric coat protein (e.g., a fiber, hexon pIX, pIIIa, or penton protein), which differs from the wild-type (i.e., native) coat protein by the introduction of a nonnative amino acid sequence, preferably at or near the carboxyl terminus. Preferably, the nonnative amino acid sequence is inserted into or in place of an internal coat protein sequence. One of ordinary skill in the art will understand that the nonnative amino acid sequence can be inserted within the internal coat protein sequence or at the end of the internal coat protein sequence. The resultant chimeric viral coat protein is able to direct entry into cells of the adenoviral vector comprising the coat protein that is more efficient than entry into cells of a vector that is identical except for comprising a wild-type adenoviral coat protein rather than the chimeric adenoviral coat protein. Preferably, the chimeric adenovirus coat protein binds a novel endogenous binding site present on the cell surface that is not recognized, or is poorly recognized, by a vector comprising a wild-type coat protein. One direct result of this increased efficiency of entry is that the adenovirus can bind to and enter numerous cell types which an adenovirus comprising wild-type coat protein typically cannot enter or can enter with only a low efficiency.

In another embodiment of the invention, the adenoviral vector comprises a chimeric virus coat protein not selective for a specific type of eukaryotic cell. The chimeric coat protein differs from the wild-type coat protein by an insertion of a nonnative amino acid sequence into or in place of an internal coat protein sequence. In this embodiment, the chimeric adenovirus coat protein efficiently binds to a broader range of eukaryotic cells than a wild-type adenovirus coat, such as described in International Patent Application WO 97/20051.

Specificity of binding of an adenovirus to a given cell can also be adjusted by use of an adenovirus comprising a short-shafted adenoviral fiber gene, as discussed in U.S. Pat. No. 5,962,311. Use of an adenovirus comprising a short-shafted adenoviral fiber gene reduces the level or efficiency of adenoviral fiber binding to its cell-surface receptor and increases adenoviral penton base binding to its cell-surface receptor, thereby increasing the specificity of binding of the adenovirus to a given cell. Alternatively, use of an adenovirus comprising a short-shafted fiber enables targeting of the adenovirus to a desired cell-surface receptor by the introduction of a nonnative amino acid sequence either into the penton base or the fiber knob.

In yet another embodiment, the nucleic acid residues encoding amino acid residues associated with native substrate binding can be changed, supplemented or deleted (see, e.g., International Patent Application Publication WO 00/15823; Einfeld et al., J. Virol., 75(23), 11284-11291 (2001); and van Beusechem et al., J. Virol., 76(6), 2753-2762 (2002)), such that the adenoviral vector incorporating the mutated nucleic acid residues (or having the fiber protein encoded thereby) is less able to bind its native substrate. For example, the native CAR and integrin binding sites of a serotype 5 or serotype 2 adenoviral vector, such as the knob domain of the adenoviral fiber protein and an Arg-Gly-Asp (RGD) sequence located in the adenoviral penton base, respectively, can be removed or disrupted. Any suitable amino acid residue(s) of a fiber protein that mediates or assists in the interaction between the knob and CAR can be mutated or removed, so long as the fiber protein is able to trimerize. Similarly, amino acids can be added to the fiber knob as long as the fiber protein retains the ability to trimerize. Suitable residues include amino acids within the exposed loops of the fiber protein, such as, for example, the AB loop, the DE loop, and the FG loop of the serotype 5 fiber knob domain, which are further described in, for example, Roelvink et al., Science, 286,1568-1571(1999), and U.S. Pat. No. 6,455,314. Any suitable amino acid residue(s) of a penton base protein that mediates or assists in the interaction between the penton base and integrins can be mutated or removed. Suitable residues include, for example, one or more of the five RGD amino acid sequence motifs located in the hypervariable region of the Ad5 penton base protein (as described, for example, U.S. Pat. No. 5,731,190). The native integrin binding sites on the penton base protein also can be disrupted by modifying the nucleic acid sequence encoding the native RGD motif such that the native RGD amino acid sequence is conformationally inaccessible for binding to the xv integrin receptor, such as by inserting a DNA sequence into or adjacent to the nucleic acid sequence encoding the adenoviral penton base protein. Preferably, the adenoviral vector comprises a fiber protein and a penton base protein that do not bind to CAR and integrins, respectively. Alternatively, the adenoviral vector comprises fiber protein and a penton base protein that bind to CAR and integrins, respectively, but with less affinity than the corresponding wild type coat proteins. The adenoviral vector exhibits reduced binding to CAR and integrins if a modified adenoviral fiber protein and penton base protein binds CAR and integrins, respectively, with at least about 5-fold, 10-fold, 20-fold, 30-fold, 50-fold, or 100-fold less affinity than a non-modified adenoviral fiber protein and penton base protein of the same serotype.

Although preferred, native binding of an adenovirus to host cells need not be ablated. In some instances, such as use of an adenoviral vector to deliver an antigen coding sequence to host cells, the broad host range of adenovirus can be advantageous.

An adenoviral vector also can comprise a chimeric coat protein comprising a non-native amino acid sequence that binds a substrate (i.e., a ligand). The non-native amino acid sequence of the chimeric adenoviral coat protein allows an adenoviral vector comprising the chimeric coat protein to bind and, desirably, infect host cells not naturally infected by the corresponding adenovirus without the non-native amino acid sequence (i.e., host cells not infected by the corresponding wild-type adenovirus), to bind to host cells naturally infected by the corresponding adenovirus with greater affinity than the corresponding adenovirus without the non-native amino acid sequence, or to bind to particular target cells with greater affinity than non-target cells. A “non-native” amino acid sequence can comprise an amino acid sequence not naturally present in the adenoviral coat protein or an amino acid sequence found in the adenoviral coat but located in a non-native position within the capsid. By “preferentially binds” is meant that the non-native amino acid sequence binds a receptor, such as, for instance, ανβ3 integrin, with at least about 3-fold greater affinity (e.g., at least about 5-fold, 10-fold, 15-fold, 20-fold, 25-fold, 35-fold, 45-fold, or 50-fold greater affinity) than the non-native ligand binds a different receptor, such as, for instance, ανβ1 integrin.

The non-native amino acid sequence can be conjugated to any of the adenoviral coat proteins to form a chimeric coat protein. Therefore, for example, the non-native amino acid sequence can be conjugated to, inserted into, or attached to a fiber protein, a penton base protein, a hexon protein, proteins IX, VI, or IIIa, etc. The sequences of such proteins, and methods for employing them in recombinant proteins, are well known in the art (see, e.g., U.S. Pat. Nos. 5,543,328; 5,559,099; 5,712,136; 5,731,190; 5,756,086; 5,770,442; 5,846,782; 5,962,311; 5,965,541; 5,846,782; 6,057,155; 6,127,525; 6,153,435; 6,329,190; 6,455,314; 6,465,253; and 6,576,456; U.S. Patent Application Publication 2001/0047081 and 2003/0099619; and International Patent Applications WO 96/07734, WO 96/26281, WO 97/20051, WO 98/07877, WO 98/07865, WO 98/40509, WO 98/54346, WO 00/15823, WO 01/58940, and WO 01/92549). The coat protein portion of the chimeric coat protein can be a full-length adenoviral coat protein to which the ligand domain is appended, or it can be truncated, e.g., internally or at the C- and/or N-terminus. The coat protein portion need not, itself, be native to the adenoviral vector. For example, the coat protein can be an adenoviral serotype 4 (Ad4) fiber protein incorporated into an adenoviral serotype 5 vector, wherein the native CAR binding motif of the Ad4 fiber is preferably ablated. However modified (including the presence of the non-native amino acid), the chimeric coat protein preferably is able to incorporate into an adenoviral capsid as its native counterpart coat protein. Once a given non-native amino acid sequence is identified, it can be incorporated into any location of the virus capable of interacting with a substrate (i.e., the viral surface). For example, the ligand can be incorporated into the fiber, the penton base, the hexon, protein IX, VI, or IIIa, or other suitable location. Where the ligand is attached to the fiber protein, preferably it does not disturb the interaction between viral proteins or fiber monomers. Thus, the non-native amino acid sequence preferably is not itself an oligomerization domain, as such can adversely interact with the trimerization domain of the adenovirus fiber. Preferably the ligand is added to the virion protein, and is incorporated in such a manner as to be readily exposed to the substrate (e.g., at the N- or C-terminus of the protein, attached to a residue facing the substrate, positioned on a peptide spacer to contact the substrate, etc.) to maximally present the non-native amino acid sequence to the substrate. Ideally, the non-native amino acid sequence is incorporated into an adenoviral fiber protein at the C-terminus of the fiber protein (and attached via a spacer) or incorporated into an exposed loop (e.g., the HI loop) of the fiber to create a chimeric coat protein. Where the non-native amino acid sequence is attached to or replaces a portion of the penton base, preferably it is within the hypervariable regions to ensure that it contacts the substrate. Where the non-native amino acid sequence is attached to the hexon, preferably it is within a hypervariable region (Miksza et al., J. Virol., 70(3), 1836-44 (1996)). Use of a spacer sequence to extend the non-native amino acid sequence away from the surface of the adenoviral particle can be advantageous in that the non-native amino acid sequence can be more available for binding to a receptor and any steric interactions between the non-native amino acid sequence and the adenoviral fiber monomers is reduced.

The non-native amino acid sequence can bind a particular cellular receptor present on a narrow class of cell types (e.g., tumor cells, cardiac muscle, skeletal muscle, smooth muscle, etc.) or a broader group encompassing several cell types. In other embodiments (e.g., to facilitate purification or propagation within a specific engineered cell type), a non-native amino acid (e.g., ligand) can bind a compound other than a cell-surface protein. Thus, the ligand can bind blood- and/or lymph-borne proteins (e.g., albumin), synthetic peptide sequences such as Polyamino acids (e.g., Polylysine, Polyhistidine, etc.), artificial peptide sequences (e.g., FLAG), and RGD peptide fragments (Pasqualini et al., J. Cell. Biol., 130, 1189 (1995)).

Examples of suitable non-native amino acid sequences and their substrates include, but are not limited to, short (e.g., 6 amino acids or less) linear stretches of amino acids recognized by integrins, as well as Polyamino acid sequences such as Polylysine, Polyarginine, etc. Inserting multiple lysines and/or arginines provides for recognition of heparin and DNA. Suitable non-native amino acid sequences for generating chimeric adenoviral coat proteins are further described in U.S. Pat. No. 6,455,314 and International Patent Application WO 01/92549.

Preferably, the adenoviral coat protein comprises a non-native amino acid sequence that binds ανβ3, ανβ5, or ανβ6 integrins. Adenoviral vectors displaying ligands specific for ανβ3 integrin, such as an RGD motif, infect cells with a greater number of ανβ3 integrin moieties on the cell surface compared to cells that do not express the integrin to such a degree, thereby targeting the vectors to specific cells of interest.

In another embodiment of the invention, the adenoviral vector can comprise a chimeric fiber protein comprising an amino acid sequence (e.g., a non-native amino acid sequence) comprising an RGD motif including, but not limited to, CRGDC (SEQ ID NO: 1), CXCRGDCXC (SEQ ID NO: 2), wherein X represents any amino acid, and CDCRGDCFC (SEQ ID NO: 3). The RGD motif can be inserted into the adenoviral fiber knob region, preferably in an exposed loop of the adenoviral knob, such as the HI loop. The RGD amino acid sequence can replace a region of the HI loop, or can be inserted into the HI loop without removal of native amino acids. The RGD motif also can be appended to the C-terminus of the adenoviral fiber protein, optionally via a spacer sequence. The spacer sequence preferably comprises between one and two-hundred amino acids, and can (but need not) have an intended function. In one embodiment, the chimeric fiber protein recognizes a coxsackievirus and adenovirus receptor (CAR). Ideally, native CAR binding of the fiber protein is not affected by mutation or modification of the fiber protein. In addition, the adenoviral vector can comprise an adenoviral coat wherein penton base proteins retain their ability to bind integrins. However, as discussed herein, native binding by the penton base proteins of the adenoviral coat protein can be ablated if desired. In another embodiment, the RGD motif preferably is flanked by one or two sets of cysteine residues.

An adenoviral vector can comprise a chimeric virus coat protein not selective for a specific type of eukaryotic cell. The chimeric coat protein differs from a wild-type coat protein by an insertion of a nonnative amino acid sequence into or in place of an internal coat protein sequence, or attachment of a non-native amino acid sequence to the N- or C-terminus of the coat protein. For example, a ligand comprising about five to about nine lysine residues (preferably seven lysine residues) is attached to the C-terminus of the adenoviral fiber protein via a non-coding spacer sequence. In this embodiment, the chimeric virus coat protein efficiently binds to a broader range of eukaryotic cells than a wild-type virus coat, such as described in International Patent Application WO 97/20051.

Of course, the ability of an adenoviral vector to recognize a potential host cell can be modulated without genetic manipulation of the coat protein. For instance, complexing an adenovirus with a bispecific molecule comprising a penton base-binding domain and a domain that selectively binds a particular cell surface binding site enables one of ordinary skill in the art to target the vector to a particular cell type.

Replication-deficient adenoviral vectors are typically produced in complementing cell lines that provide gene functions not present in the replication-deficient adenoviral vectors, but required for viral propagation, at appropriate levels in order to generate high titers of viral vector stock. Desirably, the complementing cell line comprises, integrated into the cellular genome, adenoviral nucleic acid sequences which encode gene functions required for adenoviral propagation. A preferred cell line complements for at least one and preferably all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA Polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORP of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially vaccination purposes. The lack of RCA in the vector stock avoids the replication of the adenoviral vector in non-complementing cells. Construction of such a complementing cell lines involve standard molecular biology and cell culture techniques, such as those described by Sambrook et al., supra, and Ausubel et al., supra).

Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application Publication WO 97/00326, and U.S. Pat. Nos. 5,994,128 and 6,033,908), and 293-ORF6 cells (described in, e.g., International Patent Application Publication WO 95/34671 and Brough et al., J. Virol., 71, 9206-9213 (1997)). Additional complementing cells are described in, for example, U.S. Pat. Nos. 6,677,156 and 6,682,929, and International Patent Application Publication WO 03/20879. In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the desired adenoviral vector. Helper virus is often engineered to prevent packaging of infectious helper virus. For example, one or more replication-essential gene functions of the E1 region of the adenoviral genome are provided by the complementing cell, while one or more replication-essential gene functions of the E4 region of the adenoviral genome are provided by a helper virus.

Suitable modifications to an adenoviral vector are described in U.S. Pat. Nos. 5,543,328; 5,559,099; 5,712,136; 5,731,190; 5,756,086; 5,770,442; 5,846,782; 5,871,727; 5,885,808; 5,922,315; 5,962,311; 5,965,541; 6,057,155; 6,127,525; 6,153,435; 6,329,190; 6,455,314; 6,465,253; 6,576,456; 6,649,407; 6,740,525, and International Patent Applications WO 95/02697, WO 95/16772, WO 95/34671, WO 96/07734, WO 96/22378, WO 96/26281, WO 97/20051, WO 98/07865, WO 98/07877, WO 98/40509, WO 98/54346, WO 00/15823, WO 01/58940, and WO 01/92549. Similarly, it will be appreciated that numerous adenoviral vectors are available commercially. Construction of adenoviral vectors is well understood in the art. Adenoviral vectors can be constructed and/or purified using methods known in the art (e.g., using complementing cell lines, such as the 293 cell line, Per.C6 cell line, or 293-ORF6 cell line) and methods set forth, for example, in U.S. Pat. Nos. 5,965,358; 5,994,128; 6,033,908; 6,168,941; 6,329,200; 6,383,795; 6,440,728; 6,447,995; and 6,475,757; U.S. Patent Application Publication 2002/0034735 A1, and International Patent Applications WO 98/53087, WO 98/56937, WO 99/15686, WO 99/54441, WO 00/12765, WO 01/77304, and WO 02/29388, as well as the other references identified herein.

The following examples further illustrate the invention but, of course, should not be construed as in any way limiting its scope.

### EXAMPLE 1

This example demonstrates the production of a composition comprising four adenoviral vectors each encoding a different HIV antigen.

Adenoviral vectors were constructed using a rapid vector construction system (AdFAST™, GenVec, Inc.). AdFAST™ was used to generate four adenoviral vectors each of which express one of the four HIV antigens: gp140(clade A), gp140(clade B)dv12, gp 140(clade C), and GagPol (clade B). Expression of the antigen was driven by the cytomegalovirus (CMV) immediate-early promoter. The GV11 adenoviral backbone was chosen to reduce the risk of replication-competent adenovirus (RCA) generation during clinical production. The GV11 backbone contains deletions of the essential E1 and E4 regions, as well as a partial E3 deletion that render the adenoviral vector replication-deficient.

AdtGagPol(B).11Plasmid

A synthetic Polyprotein-encoding version of the Gag/Pol genes using codons optimized for expression in human cells was created using sequences of the Gag and Pol proteins from an HIV-1 clade B were used to create. The synthetic Gag gene was from HIV-1 clade B strain HXB2 (GenBank accession number K03455), and the synthetic Pol gene (Pol/h) was from HIV-1 clade B NL4-3 (GenBank accession number M19921). The Pol gene was non-functional because it was present as a fusion protein comprising reverse transriptase, protease, and integrase proteins. Point mutations were introduced in the nucleic acid sequences encoding the protease and reverse transcriptase genes of the plasmid. The protease modification prevented processing of the Pol gene product, and reduced the potential for functional protease, reverse transcriptase, and integrase enzymatic activity. No modifications were made to the Gag protein. The nucleic acid sequence encoding the Gag/Pol fusion Polyprotein was subcloned using standard recombinant DNA techniques into an expression cassette in an E1 -shuttle plasmid for insertion into the adenoviral vector.

Adgp140(A).11D Plasmid

A synthetic version of the HIV-1 clade A gene gp140delCFI using codons altered for expression in human cells was created using the protein sequence of the Envelope Polyprotein (gp 160) from HIV-1 clade A strain 92rw020 (CCR5-tropic, GenBank accession number U08794). In this regard, plasmids expressing the HIV-1 gene were made synthetically with sequences designed to disrupt viral RNA structures that limit protein expression by using codons typically found in human cells. The nucleic acid sequence encoding the clade A gp140delCFI gene was subcloned using standard recombinant DNA techniques into an expression cassette in an E1-shuttle plasmid for insertion into the adenoviral vector.

Adtgp140dv12(B).11D Plasmid

A synthetic version of the HIV-1 clade B gene X4gp 160/h using codons optimized for expression in human cells was generated using the protein sequence of the Envelope Polyprotein (gp160) from HIV-1 clade B strain HXB2 (X4-tropic, GenBank accession number K03455). To produce a CCR5-tropic version of the Envelope protein R5gp160/h, the region encoding HIV-1 Envelope Polyprotein amino acids 275 to 361 from HIV-1 strain X4gp260/h was replaced with the corresponding region from the BaL strain of HIV-1 (GenBank accession number M68893). The full-length CCR5-tropic version of the Envelope protein gene from pR5gp160/h was terminated after the codon for amino acid 680. The truncated Env glycoprotein (gp140) contains the entire surface protein and the ectodomain of gp41 including the fusion domain, and regions important for oligomer formation, specifically two helical coiled coil motifs. The Env V1 and V2 loops were deleted to improve the stability and yield of the vector in the producer cell line. Two additional amino acids were incorporated immediately after the deletion due to creation of a restriction enzyme site. The nucleic acid sequence encoding the gp140dv12 gene was subcloned standard recombinant DNA techniques into an expression cassette in an E1-shuttle plasmid for insertion into the adenoviral vector.

Adg140(C).11D Plasmid

A synthetic version of the HIV-1 clade C gene gp140delCFI using codons optimized for expression in human cells using the protein sequence of the Envelope Polyprotein gp140delCFI from HIV-1 strain 97ZA012 (CCR5-tropic, GenBank accession number AF286227). The nucleic acid sequence encoding the synthetic gp140delCFI gene was subcloned using standard recombinant DNA techniques into an expression cassette in an E1-shuttle plasmid for insertion into the adenoviral vector.

Adenoviral Vectors

The four E1-shuttle plasmids, AdtGagPol(B).11D, Adgp140(A).11D, Adtgp140dv12(B).11D, and Adgp140(C).11D were recombined in E. coli BjDE3 bacteria with the GV11 adenovector based AdFAST™ plasmid pAdE1(BN)E3(10)E4(TIS1) to generate the adenoviral vector plasmids. The replication-deficient adenoviral vectors AdtGagPol(B).11D, Adgp140(A).11D, Adtgp140dv12(B).11D, and Adgp140(C).11D were then generated by introducing the adenoviral vector plasmids into the packaging cell line, 293-ORF6.

Adenoviral Vector Composition

The four adenoviral vector constructs were purified and dialyzed against a final formulation buffer (FFB; 10 mM Tris pH 7.8,75 mM NaCl, 5% Trehalose, 25 ppm Polysorbate 80, 1 mM MgCl₂) custom manufactured at BioWhittaker (Frederick, Md.). The adenoviral vector composition, designated VRC-HIVADV014-00-VP, was prepared from a blend of the each of the four adenoviral vectors at a 3:1:1:1 ratio by weight of AdtGagPol(B).11D, Adgp140(A).11D, Adtgp 140dv12(B).11D, Adgp140(C).11D, respectively.

### EXAMPLE 2

This example demonstrates the biodistribution of an adenoviral vector composition administered to a mammal.

A single-dose biodistribution study using intramuscular injections delivered by a needle and syringe was conducted in New Zealand White rabbits to evaluate the distribution of the adenoviral vector composition VRC-HIVADV014-00-VP. The vector composition was administered as a single dose to rabbits (0.95×10¹¹ pu), and tissues were tested for the presence of adenoviral vectors at 9, 61, and 91 days post vector administration.

Tissues were tested for the presence of the adenoviral vector using a GLP validated Taqmanm Polymerase chain reaction (PCR), developed and qualified to detect a specific target sequence in each of the four different adenoviral vectors of VRC-HIVADV014-00-VP. The assay detects an amplicon from each of the adenoviral vectors. The 5′-PCR primers, 3′-PCR primers and fluorescently labeled probes span regions containing the insert, Polylinker, and promoter. The lower limit of detection for this assay was 10 copies of VRC-HIVADV014-00-VP DNA, and the lower limit of quantification for the assay was 50 copies of VRC-HIVADV014-00-VP DNA.

The PCR data from the three timepoints showed the presence of VRC-HIVADV014-00-VP DNA in the injection site (subcutis and muscle) and liver at 9 and 61 days post administration and spleen at all timepoints. The number of copies and the number of positive tissues decreased considerably between study day (SD) 9 and SD 61 for tissues with positive findings, and between SD 61 and SD 91 for the liver and injection site. No clinical signs of toxicity or gross lesions were observed.

### EXAMPLE 3

This example demonstrates the immunogenicity of an adenoviral vector composition administered to a mammal.

The adenoviral vector composition VRC-HIVADV014-00-VP was administered a single dose (1×10¹¹ pu) to mice and twice administered to rabbits. Tissues were analyzed for immunogenicity at 4 weeks post administration for mice, and at 36 days post administration for rabbits.

Cellular immune responses were tested by the interferon gamma (IFN-γ) ELISPOT assay and the flow cytometry-based intracellular cytokine staining (ICS) assay. The IFN-γ ELISPOT quantitatively measures the production of IFN-γ by peripheral blood mononuclear cells (PBMC) from immunized animals. The cells were exposed in vitro to HIV-1 antigens (i.e., a series of short, overlapping peptides that span the length of the protein expressed in the adenoviral vector). The IFN-γ molecules produced by antigen-sensitized T-lymphocytes are bound to antibodies coating an assay plate and may be counted calorimetrically as spot forming cells (SFC) by using an alkaline phosphatase conjugated read-out system. Similarly, the ICS assay uses a flow cytometry-based system to measure IFN-γ (and sometimes additional cytokines) produced by antigen-stimulated cells. In this system, the stimulated cells are further characterized by phenotypic lymphocyte markers, allowing for precise quantification of the type of cells (for example CD4+ or CD8+ T-lymphocytes) responding to the vaccine antigens. Humoral immune responses were measured using ELISA assays or a modified assay where the antigens expressed by the adenoviral vectors were bound to the test plate using a lectin capture system.

Immunization with VRC-HIVADV014-00-VP elicited humoral and cellular immune responses in mice, and elicited humoral immune responses in rabbits.

### EXAMPLE 4

This example demonstrates the biodistribution of an adenoviral vector composition administered to a mammal.

Male and female New Zealand White rabbits, approximately 15 weeks old, were divided into two treatment groups. Group 1 consisted of three rabbits of each sex, and group 2 consisted of 15 rabbits of each sex, for a total of 36 rabbits. Group 1 animals received a single intramuscular injection (right thigh muscle) of final formulation buffer (FFB) (0.5 mL/animal) using a needle and syringe on study day (“SD”) 1. Group 2 animals received a single intramuscular injection (right thigh muscle) of a 1.0×10¹¹ pu dose of VRC-HIVADV014-00-VP.

Animals were observed at least twice daily for moribundity and mortality and clinical signs of toxicity (cageside). A detailed examination was performed at the time animals were weighed (pre-treatment, weekly thereafter, and at necropsy) in lieu of the cageside observations. Clinical signs evaluated included, but were not limited to, skin and fur characteristics, eye and mucus membranes, respiratory, circulatory, autonomic and central nervous systems, and somatomotor and behavior patterns.

Five animals per sex from the test group (Group 2) and one animal per sex from the vehicle control group (Group 1) were sacrificed on study days 9, 61 and 91. Prior to euthanasia, 0.6 mL of blood was collected by puncture of the medial auricular artery into sterile ethylene diamine tetra-acetic acid (EDTA) tubes. Each animal was euthanized by Nembutal sodium injection and exsanguinated. The following organs were collected from each animal using a clean set of instruments for each organ collected: blood, gonads, heart, lung, liver, kidney, lymph nodes, spleen, thymus, subcutis and thigh muscle (at injection site), bone marrow (from femur on side of injection) and brain. The tissues were immediately placed in sterile vials, snap-frozen in liquid nitrogen, and stored at −75°±10° C.

An adenoviral vector-specific PCR assay (Taqman™ Polymerase Chain Reaction) was used to detect the presence of the four adenoviral vectors in each tissue sample. The lower limit of detection of the assay was 10 copies of target/Ag DNA and the lower limit of quantification was 50 copies of target/μg DNA. Samples that were above the lower limit of detection but below the lower limit of quantification were designated non-quantifiable (NQ). The PCR evaluations were taken from samples harvested on study days 9, 61, and 91. A summary of the tissues exhibiting positive biodistribution results is set forth in Table 1.

No treatment related changes in mortality, clinical signs of toxicity, body weights, or body weight changes were observed. Food consumption in the male group receiving VRC-HIVADV014-00-VP was decreased during the 24-hour period following the injection, but returned to normal after that period.

TABLE 1

| | Marrow | Liver | Spleen | Subcutis | Muscle |
|---|---|---|---|---|---|
| Day 9 | | | | | |
| # positives | 1/10 | 9/10 | 10/10 | 5/10 | 4/10 |
| Avg. copy # | 23 | 945 | 1934 | 8088 | 2751 |
| Day 61 | | | | | |
| # positives | 0/10 | 2/10 | 6/10 | 2/10 | 0/10 |
| Avg. copy # | N/A | 118 | 113 | 232 | N/A |
| Day 91 | | | | | |
| # positives | 0/10 | 0/10 | 5/10 | 0/10 | 0/10 |
| Avg. copy # | N/A | N/A | 124 | N/A | N/A |

The results of this example demonstrate that the composition comprising multiple adenoviral vectors transduces a variety of tissues while exhibiting minimal toxicity.

### EXAMPLE 5

This example demonstrates the immunogenicity of an adenoviral vector composition administered to a mammal.

Two groups of female BALB/c mice were immunized with either an empty adenoviral vector or the VRC-HIVADV014-00-VP adenoviral vector composition diluted in normal saline. Specifically, five mice received an intramuscular injection of 1×10¹⁰ pu/animal of empty adenoviral vector, and ten mice received an intramuscular injection of 1×10¹⁰ pu/animal of VRC-HIVADV014-00-VP. The total volume injected for each mouse was 200 μL. Ten days after the injection, the mice were bled and sera were collected and stored at 4° C. until tested. Spleens were removed aseptically, gently homogenized to a single-cell suspension, washed, and resuspended to a final concentration of 10⁶ cells/mL.

96-well ELISA plates were coated with 100 μL/well of Lectin-Galanthaus Nivalis (Sigma) and incubated overnight at 4° C. The lectin was removed and each well was blocked with 200 μL PBS containing 10% fetal bovine serum (FBS) for 2 hours at room temperature. The plates were washed twice with PBS containing 0.2% Tween-20 (PBS-T), and 50 μL of a 1:4 dilution of protein supernatant (˜1 μg/mL) from 293 cells was added to each well. The supernatant was prepared from 293 cells transfected with DNA plasmids expressing the same HIV-1 clade A, B and C Envelope antigens as the adenoviral vector construct. Total protein from extracts of 293 cells transfected with empty p2000 vector was used as a negative control.

The plates were incubated for one hour at room temperature and washed four times with PBS-T. 50 μL of either control serum (from mice immunized with the control plasmid p2000) or serum from the test plasmid vaccinated mice were added in four-fold serial dilutions to each well, beginning at a dilution of 1:100. The plates were incubated for 1 hour at room temperature, washed, and 50 μL of horseradish peroxidase-conjugated goat antimouse IgG was added to each well. The plates were incubated for 1 hour at room temperature, washed, and 50 μL of substrate (Fast o-Phenylenediamine dihydrochloride, Sigma) were added to each well. The plates were then incubated for 30 minutes at room temperature. The reaction was stopped by the addition of 50 μL of 1(N) H₂SO₄, and optical density was read at 450 nm.

Harvested spleen cells (10⁶ cells/peptide pool) were stimulated for 6 hours. The last five hours of stimulation occurred in the presence of 10 μg/mL brefeldin A (Sigma), with peptide pools having the same amino acid sequences as those expressed by the adenoviral vectors. All peptides used were 15-mers overlapping by 11 amino acids that spanned the complete sequence of the genes tested. Cells were permeabilized, fixed and stained with monoclonal antibodies (rat anti-mouse cell surfaces antigens CD3, CD4 and CD8 (Phanningen)), followed by multiparametric flow cytometry to detect the IFN-γ and TNF-α positive cells in the CD4+ or CD8+ T-cell population. Statistical analyses in observed CD4+ and CD8+ responses between control plasmid-vaccinated and test article-vaccinated mice were performed by the Mann-Whitney test using Prism 3.0 software (San Diego, Calif.).

HIV-1-specific cellular immune responses in vaccinated mice were demonstrated by intracellular flow cytometry. Assuming a frequency of greater than 0.1% cytokine producing cells represented a positive result, then CD4+ responses were observed in 3/10 (Gag), 7/10 (Pol), 8/10 (Env-A), 10/10 (Env-B), and 9/10 (Env-C) mice. CD8+ responses were observed in 9/10 (Gag), 10/10 (Pol), 6/10 (Env-A), 6/10 (Env-B), and 7/10 (Env-C) mice. All mice had demonstrable antibody titers (measured by ELISA) to HIV-1 proteins following immunization with VRC-HIVADV014-00-VP.

These results demonstrate that the adenoviral vector composition elicited an immune response in mice.

### EXAMPLE 6

This example demonstrates the immunogenicity of an adenoviral vector composition administered to a mammal.

VRC-HIVADV014-00-VP (1×10¹¹ pu) was administered intramuscularly by needle and syringe to a group of 20 rabbits (Group 2), and an equal sized placebo group was used as a control (Group 1). A third group of rabbits (Group 3) was administered a primer composition (VRC-HIVDNA009-00-VP) (4 mg) comprising six plasmids each encoding a clade B Gag, clade B Pol, clade B nef, and Env gp145 from clades A, B and C, respectively. The clade B Pol plasmid also encoded a fusion protein comprising reverse transriptase, protease, and integrase proteins. Point mutations were introduced in the nucleic acid sequences encoding the protease and reverse transcriptase genes of the plasmid, which rendered the reverse transcriptase, protease, and integrase proteins non-functional. Following administration of the primer composition, a dose of VRC-HIVADV014-00-VP (1×10¹¹ pu) was administered to the rabbits of Group 3. Group 3 animals were compared to an equal sized placebo group (Group 4).

Following immunization, humoral immune responses were assessed by an ELISA assay. Specifically, plasmids produced at the Vaccine Research Center, National Institutes of Health (Bethesda, Md.) (VRC) (i.e., plasmid nos. 5304, 2801, and 5308) which code for HIV-Env A, B, and C, respectively were expressed in 293 cells and purified for the major protein product. Optimized concentrations of the recombinant antigens were coated on microtiter plates and kept at 4° C. overnight. The microtiter plates were washed and blocked with 20% FBS/1% BSA buffered solution and incubated. Duplicate wells of serial dilutions of the rabbit sera were incubated followed by Biotin labeled goat and rabbit, Streptavidin-HRPO, and TMB substrate. Color development was stopped and plates were read within 30 minutes at 450 nm, with the reported result based upon the average of duplicate wells.

All serum samples from rabbits in Group 1 and prebleeds for Group 2 exhibited low raw optical densities (OD), with an average OD± standard deviation of 0.159±0.105 (n=480) at dilutions of 1:100 and 1:1000. All samples from Group 2 rabbits at day 24 post administration exhibited evidence of seroconversion at serum dilutions of 1:1000. Specifically, raw optical densities for all antigens were greater than 0.21, with the average OD± standard deviation of 2.71±1.07 (n=160). All rabbits in Group 2 exhibited detectable antibody concentrations for HIV-ENV-A, ENV-B, ENV-C and GAG.

All samples from rabbits in Group 3 and prebleeds for Group 4 animals exhibited low raw optical densities (OD), with the average prevaccination OD± standard deviation of 0.099±0.065 (Group 3, n=160 samples) and 0.129±0.138 (Group 4, n=160 samples). In addition, there were very high OD values for all antigens post vaccination for rabbits in Group 4. While some rabbits in Group 4 exhibited higher OD values pre-vaccination, elevated OD values were observed at day 108 (OD=3.529±0.812), which is indicative of induced immune responses.

This example demonstrates the ability of the inventive method to induce an immune response against HIV in mammals.

### EXAMPLE 7

This example demonstrates the immunogenicity of an adenoviral vector composition administered alone or as part of a DNA prime/adenovirus boost regimen in a mammal.

Outbred adult rhesus monkeys (Macaca mulatta) were injected intramuscularly with an adenoviral vector encoding SrVmac239Gag/Pol and HIV-1 Env protein (single or multiclade) (1×10¹² pu or3.3×10¹¹ pu ) (VRC/NIH, Bethesda, Md.) either alone, or in combination with a mixture of research grade SIVmac 239 Gag/Pol-nef plasmid and single or multiclade HIV-1 Env plasmids (VRC/NIH, Bethesda, Md.). In each case, vaccine materials were mixed together in sterile saline and delivered as two 0.5 mL injections in the quadriceps muscles using a No.3 Biojector syringe (Bioject). Animals were immunized at weeks 0, 8, and 26 for the adenoviral vector alone. For the DNA/adenoviral vector prime-boost regimen, monkeys were administered plasmid at weeks 0, 4, 8 and adenoviral vector at week 26. Monkeys were bled every 2-4 weeks through week 90 post-immunization.

ELISPOT assays were utilized to monitor the emergence of vaccine-elicited T cell immune responses to multiple viral antigens. Separate assays were performed for each animal using pools of 15 amino acid peptides overlapping by 11 amino acids spanning the SIV Gag protein, pools of 20 amino acid peptides overlapping by 10 amino acids spanning the HIV-1 Env 89.6P protein (a heterologous clade B Env), and the Mamu-A*01 restricted CTL epitope peptides p11c, p41a, and p68a. 96-well multiscreen plates were coated overnight with 100 μl/well of 5 μg/mL anti-human IFN-γ (B27; BD Pharmingen) in endotoxin-free Dulbecco's PBS (D-PBS). The plates were then washed three times with D-PBS containing 0.25% Tween-20 (D-PBS/Tween), blocked for two h with D-PBS containing 5% FBS at 37° C., washed three times with D-PBS/Tween, rinsed with RPMI 1640 containing 10% FBS to remove the Tween-20, and incubated with peptide pools and 2×10⁵ PBMC in triplicate in 100 μl reaction volumes. Following an 18 h incubation at 37° C., the plates were washed nine times with D-PBS/Tween and once with distilled water. The plates were then incubated with 2 μg/mL biotinylated rabbit anti-human IFN-γ (Biosource) for two hours at room temperature, washed six times with Coulter Wash (Beckman-Coulter), and incubated for 2.5 hours with a 1:500 dilution of streptavidin-AP (Southern Biotechnology). Following five washes with Coulter Wash and one with PBS, the plates were developed with NBT/BCIP chromogen (Pierce), stopped by washing with tap water, air dried, and read using an ELISPOT reader (Hitech Instruments). Spot-forming cells (SFC) per 10⁶ PBMC were calculated. Media backgrounds consistently exhibited less than 15 spot-forming cells per 10⁶ PBMC.

Following a single adenoviral vector immunization, responses to the Gag and Env peptide pools were detected in both monkeys. Four weeks post-immunization, total spot forming cells (SFC) per 10⁶ PBMCs were 2,560 and 2,160 for monkeys Aw13 and AV83, respectively. While monkey AV83 generated enhanced Gag and Env-specific cellular immune responses following the second adenoviral vector immunization on week 8, no change in the responses of monkey Aw13 were observed. Neither monkey demonstrated augmented responses to the third adenoviral vector immunization on week 26. Cellular responses against these vaccine encoded antigens remained durable through week 52 post-immunization in monkeys Aw13 and AV83.

Cellular immune responses directed against the Gag and Env vector-encoded antigens were also analyzed by pooled peptide ELISPOT assays following immunization with a DNA prime/adenoviral vector boost regimen. Following adenoviral vector boost at week 26, cellular immune responses to the Gag and Env peptide pools increased 5-6-fold higher compared to DNA vaccination alone in monkeys Aw2P and Aw28. At Week 30, i.e., four weeks post-immunization, total SFC per 10⁶ PBMCs were 7010 and 7805 for monkeys Aw2P and Aw28, respectively. Cellular responses against these vector-encoded antigens remained durable through week 58 post-immunization, with 4265 and 3000 SFC per 10⁶ PBMC measured in monkeys Aw2P and Aw28.

To assess the contribution of antigen-specific CD4+ and CD8+ T lymphocytes in cellular immunity elicited by the adenoviral vector construct, peptide ELISPOT assays were performed using unfractionated and CD8+ T lymphocyte-depleted PBL on week 28, two weeks following the final adenoviral vector immunization. While potent cellular immune responses were measured against Gag and Env peptide pools using whole PBL, these responses were substantially reduced when CD8+ T lymphocytes were removed from the PBL population, demonstrating that immunizations with adenoviral vectors elicit potent cellular immune responses that are predominantly CD8+ T lymphocyte mediated.

A direct enzyme-linked immunosorbent assay (ELISA) was used to measure plasma titers of anti-gp120 (HIV-MN) and anti-p27 SIVmac239 antibodies (see, e.g., VanCott et al., J. Virol., 73(6), 4640-50(1999)). Both monkeys had demonstrable antibody titers (measured by ELISA) to gp140 89.6 Envelope proteins following adenoviral vector immunization. Strong homologous neutralizing antibody titers were also measured in all four immunized animals but the magnitude of the responses in the DNA prime/adenovirus boosted animals was several fold higher than those observed after adenoviral vector vaccination alone.

A flow based neutralization assay was used to measure plasma-mediated virus neutralization. Plasma samples were heat-inactivated to deplete complement proteins and tested at a 1:5 dilution. Percent neutralization mediated by week 28 and week 32 were calculated by comparison to the week 0 pre-immune plasma (see, e.g., Mascola et al., J. Virol., 76(10), 4810-21 (2002)). Neutralizing antibodies against HIV-1 89.8 Envelope antigen were also demonstrated. The magnitude of neutralizing antibody responses in the DNA prime/adenoviral vector boosted vaccinated animals was higher than in the adenoviral vector vaccinated animals.

These results show that the adenoviral vector composition can elicit an immune response in a mammal when administered alone, and that the immune response can be enhanced when the adenoviral vector composition is used as part of a DNA prime/adenovirus boost regimen in a mammal.

### EXAMPLE 8

This example demonstrates the use of the inventive method to induce protective immunity against an HIV antigen that is not present in the adenoviral vector composition or the primer composition.

Twenty-four outbred adult Indian-origin rhesus monkeys (Macaca mulatta) were injected intramuscularly with DNA constructs expressing SlVmac 239 Gag/Pol DNA, HIV-1 89.6P Env DNA (VRC/NIH, Bethesda, Md.), or HXB2/Bal Env DNA, followed by a boost administration of a recombinant adenoviral vector. Because of instability, the research grade adenoviral vector was constructed without Nef (see Letvin et al., Journal of Virology, In press).

In each case, vaccine constructs were mixed together in sterile saline and delivered as two 0.5 mL injections in the quadriceps muscles using a No.3 Biojector syringe (Bioject). DNA immunization occurred at weeks 0, 4, 8 and adenoviral vector immunization occurred at week 26 (1×10¹² pu ) for the DNA/adenoviral vector prime-boost regimen. Monkeys were bled every 2-4 weeks through week 90 post-immunization. The following four experimental groups were tested: (1) control, (2) Gag/Pol/Nef DNA and Gag/Pol adenoviral vector with no Env (mock), (3) Gag/Pol/Nef DNA and Gag/Pol adenoviral vector with SHIV-89.6P Env, or 4) Gag/Pol/Nef DNA and Gag/Pol adenoviral vector with HXB2/Bal Env.

All monkeys were challenged intravenously with monkey infectious dose 50 (MID50) SHIV-89.6P on week 38, i.e., 12 weeks following the adenoviral vector boost. Monkeys were bled every 2-4 weeks following both immunization and challenge.

Freshly isolated peripheral blood mononuclear cells (PBMC) were assessed for interferon gamma ELISPOT responses to SlVmac after in vitro exposure to peptide pools spanning the SlVmac Gag/Pol/Nef and HW-1 Env proteins. All Env-specific responses were assessed using peptides that were matched to the Env immunogen. Test systems are described in Letvin et al., supra.

ELISPOT responses from the PBMCs of all monkeys receiving experimental immunogens were robust. Cellular immunity to SIV Gag, Pol and Nef was generated in all groups of vaccinated monkeys, and to HIV-1 89.6P and HXB2/Bal Env in monkeys receiving these respective immunogens. Mean total vaccine-elicited ELISPOT responses to all viral proteins two weeks after the final plasmid DNA inoculations were 1,588±554 standard error of the mean (SEM) spot forming cells (SFC) in the mismatched Env group. Two weeks after boosting with recombinant adenoviral vectors, there was a >2.5-fold increase over the cellular immunity elicited by DNA priming alone.

Following challenge with monkey infectious dose 50 (MID50) SHIV-89.6P on week 38, a profound loss of CD4+ T lymphocyte was observed in all controls, while substantial blunting of that CD4+ T lymphocyte depletion was seen in all vaccinated animals. This blunting was most significant in the monkeys that received HIV-1 Env in addition to SIV Gag/Pol-Nef, documenting statistically significant protection against CD4+ T lymphocytes loss afforded by inclusion of Env component in the vaccine. Importantly, monkeys that received the mismatched Env immunogens showed comparable protection to those injected with the matched immunogens. The group of monkeys that received the SIV Gag/Pol/Nef+ mismatched Env immunogens also demonstrated better containment of virus, indicated by reduced viral loads.

These results show that the adenoviral vector composition can be used to elicit an immune response to HIV in a mammal.

### EXAMPLE 9

This example demonstrates the cellular immune responses elicited by an adenoviral vector composition administered as part of a DNA prime/recombinant adenovirus boost regimen in a mammal.

Outbred adult rhesus monkeys (Macaca mulatta) were injected intramuscularly with mixtures of GLP grade plasmid DNA vectors encoding SIV Gag/Pol/Nef proteins and multiclade A, B, and C HIV-1 Env proteins contained in a composition designatedVRC-HIVDNA009-00-VP. An adenoviral vector encoding SlVmac 239 Gag/Pol and an adenoviral vector encoding HIV-1 clade A, B, and C Env were used to boost.

In each case, plasmids or adenoviral vector were mixed together in sterile saline and delivered as two 0.5 mL injections in the quadricep muscles using a No. 3 Biojector syringe (Bioject). Animals were immunized at weeks 0, 4, and 8 with plasmid DNA, and week 26 with adenoviral vector. Animals were bled every 2-4 weeks through week 42. The specific prime and boost immunizations are set forth in Tables 2 and 3, respectively.

TABLE 2

| | Number | | | |
|---|---|---|---|---|
| | of | SIV Gag/Pol/Nef | | Sham |
| Group | Animals | Plasmid | HIV-1 Env Plasmid(s) | Plasmid |
| 1 | 6 | 4.5 mg | 4.5 mg (clade B) | — |
| 2 | 6 | 4.5 mg | 4.5 mg (clade C) | — |
| 3 | 6 | 4.5 mg | 1.5 mg (clade A) | — |
| | | | 1.5 mg (clade B) | |
| | | | 1.5 mg (clade C) | |
| 4 | 6 | 4.5 mg | 1.5 mg (clade B) | 3.0 mg |
| 5 | 6 | — | — | 9.0 mg |

TABLE 3

| | Number | SIV Gag/Pol | HIV-1 Env | Sham |
|---|---|---|---|---|
| | of | adenoviral | adenoviral vector(s) | adenoviral |
| Group | Animals | vector (pu) | (pu) | vector |
| 1 | 6 | 1.0 × 10¹² | 1.0 × 10¹² (clade B) | — |
| 2 | 6 | 1.0 × 10¹² | 1.0 × 10¹² (clade C) | — |
| 3 | 6 | 1.0 × 10¹² | 3.3 × 10¹¹ (clade A) | — |
| | | | 3.3 × 10¹¹ (clade B) | |
| | | | 3.3 × 10¹¹ (clade C) | |
| 4 | 6 | 1.0 × 10¹² | 3.3 × 10¹¹ (clade B) | 6.6 × 10¹¹ |
| 5 | 6 | — | — | 2.0 × 10¹² |

ELISPOT assays were utilized to monitor the emergence of vaccine-elicited T cell immune responses to multiple viral antigens. Separate assays were performed for each animal using pools of 15 amino acid peptides overlapping by 11 amino acids spanning the SIV Gag, SIV Pol, SIV Nef, HIV-1 Env clade A, HIV-1 Env clade B, and HIV-1 Env clade C proteins matching the sequences of the immunogens encoded by the adenoviral vectors. Assays were also performed using pools of 20 amino acid peptides overlapping by 10 amino acids spanning HIV-1 Env 89.6P, which is a clade B Env sequence heterologous to the immunogens encoded by the adenoviral vectors. 96-well multiscreen plates were coated overnight with 100 μl/well of 5 μg/mL anti-human IFN-γ (B27; BD Pharmingen) in endotoxin-free Dulbecco's PBS (D-PBS). The plates were then washed three times with D-PBS containing 0.25% Tween-20 (D-PBS/Tween), blocked for 2 hours with D-PBS containing 5% FBS at 37° C., washed three times with D-PBS/Tween, rinsed with RPMI 1640 containing 10% FBS to remove the Tween-20, and incubated with peptide pools and 2×10⁵ PBMC in triplicate in 100 μl reaction volumes. Following 18 hours incubation at 37° C., the plates were washed nine times with D-PBS/Tween and once with distilled water.

The plates were then incubated with 2 μg/mL biotinylated rabbit anti-human IFN-γ (Biosource) for 2 hours at room temperature, washed six times with Coulter Wash (Beckman-Coulter), and incubated for 2.5 hours with a 1:500 dilution of streptavidin-AP (Southern Biotechnology). Following five washes with Coulter Wash and one with PBS, the plates were developed with NBT/BCIP chromogen (Pierce), stopped by washing with tap water, air dried, and read using an ELISPOT reader (Hitech Instruments). Spot-forming cells (SFC) per 10⁶ PBMC were calculated, Media backgrounds were consistently less than 15 spot-forming cells per 10⁶ PBMC.

The extent of cross-clade reactivity of cellular immune responses elicited by single clade Env immunization was investigated by assessing responses in Group 1 (high clade B Env) and Group 2 (high clade C Env). For the DNA prime immunizations, monkeys received 4.5 mg Gag/Pol/Nef plasmid with 4.5 mg of Env plasmid from clade B (Group 1) or clade C (Group 2). PBMCs were tested for Env-specific cellular immune responses by pooled peptide ELISPOT assays using peptide pools from Env clade A, Env clade B, Env clade C, and Env 89.6P (a heterologous clade B Env). Monkeys in Group 1 that received the Env clade B plasmid generated responses to all Env peptide pools, demonstrating a degree of cross-clade reactivity. However, clade B peptide responses were higher than clade A or clade C responses. The DNA primed cellular immune responses of monkeys in Group 1 were dramatically augmented following the boost immunization with 1.0×10¹² pu Gag/Pol and 1.0×10¹² pu clade B Env adenoviral vector. While responses to all Env peptide pools were observed from these monkeys following the adenoviral vector boost immunization, all six animals demonstrated the highest response to clade B Env.

Similarly, monkeys in Group 2 that received the Env plasmid and adenoviral vector from clade C generated responses to all Env peptide pools. Clade C responses were higher than clade A or clade B responses in all six animals following the DNA prime immunizations and following the adenoviral vector boost. These data demonstrate that DNA prime/adenoviral vector boost immunization with single clade Env immunogens elicits Env-specific cellular immune responses with partial cross-clade reactivity, but that the highest responses were generally against the Env clade matching the immunogen.

The Env-specific cellular immune responses of monkeys in Group 4 (low clade B Env) were comparable with responses of monkeys in Group 1 (high clade B Env). Monkeys in Group 4 received 4.5 mg Gag/Po/Nef plasmid with 1.5 mg Env plasmid from clade B for the DNA prime immunizations, and 1.0×10¹² pu Gag/Pol adenoviral vector with 3.3×10¹¹ PU clade B Env adenoviral vector for the boost immunization. These observations suggest that lowering the dose of a single Env plasmid or adenoviral vector threefold does not result in major reductions in immunogenicity. Minimal background responses were observed in monkeys in Group 5 that received only sham plasmids and adenoviral vector.

The breadth and magnitude of cellular immune responses elicited by the multiclade Env immunizations were investigated by assessing responses in Group 3 (clade A+B+C Env). For the DNA prime immunizations, these monkeys received 4.5 mg Gag/Pol/Nef plasmid with 1.5 mg of each Env plasmid from Clades A, B, and C (4.5 mg Env plasmids total). Similar magnitude and broad cellular immune responses to Env clade A, B, and C were observed. These data demonstrate that the mixture of the three Env plasmids in Group 3 resulted in increased breadth without loss of magnitude of the responses, despite the fact that each Env plasmid component in Group 3 was given at the 1.5 mg rather than the 4.5 mg dose. Following the boost immunization with 1.0×10¹² pu Gag/Pol adenoviral vector and 3.3×10¹¹ pu Env adenoviral vector of each clade A, B, and C, all six monkeys demonstrated similar magnitude responses to clade A, B, and C Env peptide pools. These data demonstrate that the magnitude of each individual clade-specific response in Group 3 was comparable with the optimal clade-specific response elicited in Groups 1 and 2.

Cellular immune responses to SIV Gag and Pol were observed in all vaccinated monkeys following DNA immunization and following adenoviral vector boost. Monkeys receiving the four-component multiclade vaccine product (Group 3) elicited similar magnitude cellular immune responses to SIV Gag and SIV Pol as observed in monkeys receiving single clade Env immunogens. The four-component multiclade vaccine (Group 3) thus resulted in broader responses to all vaccine-encoded antigens without loss of immunogenicity as compared with the single clade vaccines (Groups 1, 2, and 4). Furthermore, cellular immune responses to these antigens were found to be durable following both DNA prime and adenoviral vector boost immunization.

The humoral immune responses elicited by single clade and multiclade Env immunizations were investigated by assessing Env-specific antibody titers from monkeys following adenoviral vector boost immunization. Plasma samples were tested for Env lade A, clade B, or clade C specific antibody binding activity as measured by ELISA.

Endpoint titers were determined for week 10 (post DNA) and week 40 (post adenoviral vector) as the last dilution with pre-immunization corrected optical density (OD) greater than 0.2. Wells were coated with 37.5 ng purified Env antigen overnight at 4° C. Plates were washed, and blocked (20%FBS/1%BSA buffered solution) for 1 hour at 37° C. Duplicate wells of serial dilutions of the sera were incubated 2 hours at 37° C. followed by Biotin labeled goat anti-monkey (1 hour 37° C.), streptavidin-IPO (30 minutes, room temperature (RT)), and TNB substrate (30 minutes, RT). Color development was stopped by adding sulfric acid and plates were read within 30 minutes at 450 nm, with reported results based upon the average of duplicate wells.

Monkeys in Group 1 (high clade B Env) generated antibody responses that were capable of binding to all three Env antigens, demonstrating a degree of cross-clade reactivity. While robust responses were measured against the clade B and C Env antigens, the highest antibody titers were detected against the homologous clade B Env. Monkeys in Group 4 (low clade B Env) exhibited Env-specific antibody titers that were similar in breadth and magnitude as to those measured in Group l monkeys, demonstrating that lowering the dose of Env immunogen threefold did not result in reduced immunogenicity. Monkeys in Group 2 (high clade C Env) similarly elicited antibody responses capable of recognizing all three Env antigens, but highest titers were detected against the homologous clade C Env. Monkeys immunized with the mixture of clade A, clade B, and clade C Env antigens (Group 3), however, demonstrated high magnitude antibody titers to all three Env antigens.

These data suggest that multiclade Env immunization resulted in increased breadth of the humoral immune response without a loss of immunogenicity when compared to responses elicited by single clade Env immunization.

### EXAMPLE 10

This example demonstrates the cellular and humoral immune responses elicited by an adenoviral vector composition administered as part of a DNA prime/recombinant adenovirus boost regimen in a mammal.

Outbred adult Cynomolgus macaques were injected intramuscularly with mixtures of vaccine plasmids or adenoviral vector constructs. Specifically, GLP plasmid DNA expressing Gag/Pol/Nef proteins and multiclade A, B, and C HIV-1 Env proteins contained in composition VRC-HIVDNA009-00-VP (Example 8) were used for the prime immunization. GMP grade VRC-HIVADV014-00-VP (Example 1) was used as the adenoviral vector boost.

To achieve the required volumes for the three scheduled injections in the animal study, three lots of formulated material were prepared. The three lots were combined in a 50 mL conical tube. Following inversion of the tube several times to mix, 15.6-15.7 mL of the mixture was aliquotted into each of three 50 mL conical tubes. Tubes were labeled and stored at −20° C. until distributed.

8 mg of the DNA composition was delivered intramuscularly (i.m.) at weeks 0, 4, and 8 by Biojector and 10¹¹ pu total adenoviral vector vaccine construct was delivered i.m. by needle and syringe at week 38. Animals were bled every 24 weeks through week 42.

ELISPOT assays were utilized to monitor the emergence of vaccine-elicited T cell immune responses to multiple viral antigens as described in Example 8. A direct ELISA was used to measure plasma titers of Env clade A, clade B, and clade C antibodies as described in Example 8.

Monkeys that received the DNA plasmid vaccine prime and adenoviral vector boost generated responses to clade A, B, and C Env peptide pools in all six animals following the DNA prime immunizations and following the adenoviral vector boost. Five of six animals developed antibody responses to all three Envelope antigens (clade A, B, and C). One animal developed a humoral immune responses to clade A and C Envelope only. All six monkeys had strong Env antibody responses after adenovirus boost.

These data demonstrate that the clinical DNA prime/adenoviral vector product is immunogenic and induces cellular immune responses against clade A, B, C Env as well as Gag and Pol, and antibody responses against clade A, B, and C Env as well as Gag. Adenovector boosting increases the immune responses several fold.

### EXAMPLE 11

This example demonstrates cellular immune responses elicited by recombinant adenovirus boost immunizations in Cynomolgus monkeys. Six cynomolgus macaques (Mauritius origin) were immunized once intramuscularly with a 1×10¹¹ pu dose of the adenoviral vector composition VRC-HIVADV014-00-VP (Example 1). The composition was delivered as two 0.5 mL injections in the quadricep muscles using a needle and syringe. Monkeys were bled every 2-4 weeks through week 4 post-immunization. ELISPOT assays were utilized to monitor the emergence of vaccine-elicited T cell immune responses to multiple viral antigens, as described in Example 8.

Monkeys that received the adenoviral vector generated responses to clade A, B, and C Env peptide pools in all six animals. These data demonstrate that the clinical adenoviral vector product is immunogenic and induces cellular immune responses against clade A, B, C Env, as well as Gag and Pol.

The animals were clinically evaluated by a Laboratory Animal Medicine certified veterinarian after chemical anesthesia by ketamine hydrochloride at pre-immunization (week −1), week 0 (1^{st} immunization), and 1, 2, 3, 4, 5, and 8 week time points. Serum chemistries and complete blood count were determined at weeks −1, 3, and 5. Subject animals were found to be healthy and in excellent condition at all time points evaluated. Physical examination included auscultation, palpation, and determination of body temperature, pulse, and respiration. Body temperatures, pulse, and respiration were within normal limits. A pea-sized inguinal lymph node was detected in two monkeys (CO 7422 and CO 7414) at weeks 1 and 5, respectively, on the ipsilateral side of the inoculation. White blood counts and hematocrit values were generally within normal limits and with minimal variation between pre- and post-immunization time points for all animals. Serum electrolytes, blood urea nitrogen and creatinine were also within normal limits.

All animals had aspartate aminotransferase/glutamic oxaloacetic transaminase (AST/GOT), alkaline phosphatase, and total bilbubin levels with normal limits in pre-immunization serum as well as at immunization. Animal CO 7412 had a pre-immunization alanine aminotransferase/glutamic pyruvic transaminase (ALT/GPT) of 97 U/L (normal range 0-138 U/L). The ALT was slightly elevated after immunization (177 U/L at 3 weeks, 166 U/L at 5 weeks), but within normal limits (136 U/L) at 8 weeks post immunization. Enzymes creatinine kinase and lactate dehydrogenase were minimally increased at week 5 in animals CO 7423 and CO 7420, which most likely represented ketamine induced muscle damage. The values returned to normal at week 8.

These data demonstrate that the inventive method elicited potent and broad cellular immune responses against all viral antigens in cynomolgus macaques.

### EXAMPLE 12

This example demonstrates the safety of an adenoviral vector composition administered to a mammal.

Female and male New Zealand white rabbits were administered via intramuscular injection a DNA priming construct (VRC-HIVDNA009-00-VP) and the adenoviral vector construct VRC-HIVADV014-00-VP as a boost, or VRC-HIVADV014-00-VP alone. VRC-HIVADV014-00-VP was produced as described in Example 1.

For the DNA prime/adenovirus boost method, 4 mg of VRC-HIVDNA009-00-VP or the PBS control (study day 1, 22) were administered via two intramuscular injections (0.5 mL/injection site; dose volume for each injection was not adjusted for body weight) per day of dosing into the thigh muscle (two injections spaced approximately 1 inch apart) using a Biojector 2000® Needle-Free Injection Management System™ (Bioject). Injections were administered on alternate sides for each time point. Each injection was administered at a shaved/marked site. The site was re-shaved and re-marked as needed in order to visualize the injection site.

For both the DNA prime/adenovirus boost study and the study involving only the adenoviral vector, VRC-HIVADV014-00-VP (1×10¹¹ pu) or the diluent control (VRC-DILUENT013-DIL-VP) injections were administered as two 0.5 mL injections per day of dosing into the hind thigh muscle with a needle and syringe. Each injection was administered at a shaved/marked site. The site was re-shaved and re-marked as needed in order to visualize the injection site.

Animals were randomly assigned to treatment groups. The treatment period was 22 days, and the study duration was 36 days. Injections were administered on alternate sides for each time point. 1.0 mL was administered regardless of body weight for DNA and adenovector doses and their respective controls.

Blood samples (approximately 2 mL) were isolated from all animals prior to administration of the first dose. The samples were subjected to hematology, chemistry, coagulation, and immunology analyses. Serum was isolated and stored at −75° C.±10° C. for transfer on dry ice. Some or all of these samples were analyzed for seroconversion as an indication of exposure to the test article.

Following terminal blood collection, all animals were euthanized by sodium pentobarbital or equivalent injection and exsanguinated. Animals were necropsied as close as possible to the time of sacrifice. Scheduled necropsies were conducted under the supervision of a veterinary pathologist.

All required animals were subjected to a full gross necropsy, which included examination of the external surface of the body, the injection/treatment site, all orifices, and the cranial, thoracic, and abdominal cavities and their contents. Two bone marrow smears were prepared from the sternum of each animal. Slides were air-dried, fixed in methanol, and archived for possible future evaluation. The following organs (sex appropriate) were weighed as soon as possible from all required animals at scheduled necropsy: adrenal glands, heart, lung, brain, spleen, kidneys, liver (with drained gallbladder), testes/ovaries, pituitary, thymus, uterus, and thyroids/parathyroids. Paired organs were weighed together.

All tissues from each necropsied animal were preserved in 10% neutral buffered formalin (NBF). The tissues were embedded in paraffin, sectioned, stained with hematoxylin and eosin, and examined microscopically by a board certified veterinary pathologist. Tissues from each animal and from gross lesions (from all groups) were analyzed.

Quantitative results were analyzed using the Kolmogorov-Smirnov test for normality, the Levene Median test for equal variance, and by one-way Analysis of Variance (ANOVA). If either the normality or equal variance test failed, then the analysis employed the non-parametric Kruskal-Wallis ANOVA on rank-transformed data. For parametric data, if the ANOVA indicated statistical significance among experimental groups then the Dunnett's t-test was used to delineate which groups (if any) differed from the control. For non-parametric data, if the ANOVA indicated statistical significance among experimental groups then the Dunn's test was used to delineate which groups (if any) differed from the control. The probability value of less than 0.05 (two-tailed) was used as the critical level of significance for all tests. Statistical analysis utilized SigmaStat™ Statistical Software (Jandel Scientific, San Rafael, Calif.).

For the immunization strategy involving administration of VRC-HVADV014-00-VP alone, all animals survived to the scheduled termination and no treatment-related effects were noted in the following parameters: mortality, clinical and cageside observations, Draiz observations, body weights, ophthalnology, clinical pathology, and organ weights (with exception of an increased spleen weight, which is likely an expected result of exposure to an immunostimulatory agent) or organ weight ratios. There were increased body temperatures in the treated animals 24 hours after the first injection. There was also decreased food consumption in the treated animals for the 24-48 hour period after each injection. Transient inflammation at the injection site in treated animals was observed, as was recoverable, chronic inflammation in the connective tissue around the sciatic nerve and adjacent lymphatics and blood capillaries. Transient increases in cholesterol and triglyceride levels at SD 3 were not associated with clinical symptoms or pathology, and the transient increase in CPK at SD 24 was possibly related to muscle inflammation.

For the DNA prime/adenoviral vector boost strategy, recoverable inflammation at the injection sites (observed by Draize scoring and histopathologically) and perineural tissue around the sciatic nerves (seen only histopathologically) were observed. In addition, fevers were noted in immunized rabbits in the 24 hours subsequent to the initial and in immunized females in the three hours subsequent to the second adenovector boost. Food consumption was also reduced in the 24-48 hours subsequent to each vaccination, although this resolved and did not impact body weights or weight gain in males. However, treated females did have reduced body weights and weight gains compared to control females, which became statistically significant after SD71 (body weight) and after the initial adenovector boost (weight gains), but which began to be observed as early as SD36 (during the DNA priming series).

This example demonstrates that the inventive method induces minimal toxicity in rabbits.

### EXAMPLE 13

This example demonstrates the administration of an adenoviral vector composition to humans.

A randomized, placebo-controlled, double-blinded, dose escalation study was initiated to examine safety, tolerability and immune response in humans following a single injection of VRC-HEADV014-00-VP at a dose of 1×10⁹ pu, 1×10¹⁰ pu, or 1×10¹¹ pu. Each treatment group included 12 subjects (10 vaccines; 2 placebos). The study was initiated on Jul. 19, 2004 and the study completed enrollment of 36 subjects on Nov. 10, 2004. The NIAID Intramural Data and Safety Monitoring Board (DSMB) reviewed the preliminary safety data through 14 days of follow-up prior to each dose escalation. The preliminary data indicated that VRC-HIVADV014-00-VP appears to be safe for healthy subjects at the three dose levels evaluated. The 1×10⁹ pu and 1×10¹⁰ pu dose levels were associated with less reactogenicity than the 1×10¹¹ pu dose level. In both the 1×10⁹ pu and 1×10¹⁰ pu dose groups, the local and systemic symptoms recorded on the 5-day diary card were none to mild in severity, and none of the subjects experienced fever. In the 1×10¹¹ pu dose group, four subjects reported fever on Day 1 (3 mild and 1 moderate in severity). Each of the four subjects with fever also reported moderate headache on Day 1 and three of these subjects also reported at least one other moderate systemic parameter (e.g., malaise, myalgia, and chills). Two subjects without fever reported at least one moderate systemic symptom (e.g., malaise, myalgia, and nausea). One subject in the 1×10¹¹ pu dose group reported moderate injection site pain; injection site reactogenicity was otherwise none or mild.

As of Jan. 31, 2005, there was one grade 4 (potentially life-threatening) event. There were three grade 2 (moderate) adverse events that were possibly related to vaccination. The study was blinded to vaccine vs. placebo injection assignments. The grade 4 adverse event was a seizure that occurred 64 days after study injection in a healthy subject in the 1×10¹¹ pu dose group who had a history of a single seizure three years prior to study enrollment. Given history of a prior seizure and the timing of the event more than 2 months after study injection, it seemed unlikely that the seizure was related to study agent. The grade 2 adverse events possibly related to study agent included: (1) asymptomatic neutropenia noted 21 days after study injection in a subject known to sometimes have asymptomatic low neutrophil counts prior to enrollment, (2) diarrhea (duration one day) in a different subject on the third day after study injection, and (3) steatohepatitis (fatty liver) diagnosed after extensive evaluation to identify the cause of a persistent grade 1 ALT (alanine aminotransferase) elevation that was noted starting 25 days after the study vaccination in a clinically asymptomatic subject. A hepatology consultant reported an impression that the condition likely existed prior to study enrollment. Contributing factors to the persistent grade 1 ALT may be alcohol consumption and recent weight gain. A diagnosis of steatohepatitis is overall considered to be a grade 2 condition, but as of Jan. 31, 2005, the liver function tests were not more than grade 1 in severity.

Although more reactogenicity was observed with the 1×10¹¹ pu dose, it appeared to be a well-tolerated dose and analgesic/antipyretic nonprescription medications can be self administered for relief of the short-term symptoms. A protocol-specified interim immunogenicity analysis is in progress to compare the placebo and three dosage groups. The blinded immunogenicity data suggest a dose effect with increasing immune response at higher doses. The number of subjects with vaccine-induced ELISA at study week 12 by commercial HIV-antibody assay increased from 3 in the 1×10⁹ pu group, to 6 in the 1×10¹⁰ pu group, and to 9 in the 1×10¹¹ pu group among the 12 subjects (two placebos and ten vaccine recipients) per group (study assignments were blinded). The reactogenicity data are summarized in Table 4 below.

TABLE 4

| | 10⁹ pu or | 10¹⁰ pu or | 10¹¹ pu or |
|---|---|---|---|
| | placebo | placebo | placebo |
| Reactogenicity | (N = 12) | (N = 12) | (N = 12) |
| Local Symptoms | | | |
| None | 9 (75%) | 3 (25%) | 2 (16.7%) |
| Mild | 3 (25%) | 9 (75%) | 9 (75.0%) |
| Moderate | 0 | 0 | 1 (8.3%) |
| Severe | 0 | 0 | 0 |
| Systemic Symptoms | | | |
| None | 10 (83.3%) | 4 (33.3%) | 3 (25%) |
| Mild | 2 (16.7%) | 8 (66.7%) | 3 (25%) |
| Moderate | 0 | 0 | 6 (50%) |
| Severe | 0 | 0 | 0 |

These results indicate that the inventive method is well tolerated in humans.

### EXAMPLE 14

This example demonstrates the administration of VRC-HIVADV014-00-VP to humans.

A second Phase I study of the adenoviral vector composition VRC-HIVADV014-00-VP as single agent in uninfected adult subjects is currently in progress. This blinded, dose escalation study is designed to enroll two groups of 24 subjects with low Ad5 antibody titer (<1:12), who will be randomized to VRC-HIVADV014-00-VP or placebo in a 5:1 ratio. The first group of vaccines will receive 1×10¹⁰ pu VRC-HIVADV014-00-VP and the second group will receive 1×10¹¹ pu VRC-HIVADV014-00-VP.

### EXAMPLE 15

This example demonstrates the administration of VRC-HIVADV014-00-VP to humans as a booster following immunization with a DNA molecule.

A Phase I, blinded, placebo-controlled study has been initiated, which provides a single adenoviral vector boost of VRC-HIVADV014-00-VP at 1×10¹⁰ pu (or placebo) to participants who complete a DNA injection regimen with VRC-HIVDNA009-00-VP. The adenoviral vector vaccine boost will be given at an interval of six to nine months after the initial DNA vaccination with VRC-HIVDNA009-00-VP (or placebo injection). The first participant was enrolled on Nov. 22, 2004. As of Dec. 21, 2004, 11 participants have received their boost injection. Of these participants, six have experienced mild pain and/or tenderness at the injection site. There have been no other reports of local reactogenicity events. Five participants reported either mild or moderate systemic symptoms including headache, malaise and nausea. There have been no reports of fever, no grade 3 events, and no serious adverse events.

These results indicate that the inventive method is well tolerated in humans.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” ”.including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inducing an immune response against a human immunodeficiency virus (HIV) in a mammal comprising administering to the mammal an adenoviral vector composition, wherein the adenoviral vector composition comprises one or more adenoviral vectors encoding two or more different HIV antigens, whereupon the HIV antigens are produced in the mammal and an immune response against HIV is induced.

2. The method of claim 1, wherein the adenoviral vector composition comprises two or more adenoviral vectors encoding the two or more different HIV antigens, and each adenoviral vector comprises a nucleic acid sequence that encodes at least one of the two or more different HIV antigens.

3. The method of claim 2, wherein the adenoviral vector composition comprises three or more adenoviral vectors encoding three or more different HIV antigens, and each adenoviral vector comprises a nucleic acid sequence that encodes at least one of the three or more different HIV antigens.

4. The method of claim 3, wherein the adenoviral vector composition comprises four or more adenoviral vectors encoding four or more different HIV antigens, and each adenoviral vector comprises a nucleic acid sequence that encodes at least one of the four or more different HIV antigens.

5. The method of claim 1, wherein the one or more adenoviral vectors each comprise (i) a nucleic acid sequence that encodes two or more different HIV antigens, or (ii) two or more nucleic acid sequences that each encode a different HIV antigen.

6. The method of claim 5, wherein the one or more adenoviral vectors each comprise a nucleic acid sequence that encodes two or more different HIV antigens.

7. The method of claim 5, wherein the one or more adenoviral vectors each comprise two or more nucleic acid sequences that each encode a different HIV antigen.

8. The method of any of claims 1-7, further comprising administering to the mammal a primer composition comprising one or more nucleic acid sequences that encode at least one HIV antigen that is the same as an HIV antigen encoded by an adenoviral vector of the adenoviral vector composition, wherein the administration of the primer composition is performed at least one week before the administration of the adenoviral vector composition.

9. The method of claim 8, wherein the primer composition comprises one or more nucleic acid sequences that encode two or more HIV antigens that are the same as the HIV antigens encoded by the one or more adenoviral vectors of the adenoviral vector composition.

10. The method of claim 8 or claim 9, wherein the administration of the primer composition is performed about six months to about nine months before the administration of the adenoviral vector composition.

11. The method of any of claims 8-10, wherein the primer composition comprises one or more plasmids, naked DNA molecules, or viral vectors comprising the one or more nucleic acid sequences.

12. The method of any of claims 1 -11, wherein the adenoviral vectors are replication-deficient.

13. The method of claim 12, wherein the adenoviral vectors are deficient in one or more essential gene functions of the E1 region of the adenoviral genome.

14. The method of claim 12 or 13, wherein the adenoviral vectors are deficient in one or more essential gene functions of the E4 region of the adenoviral genome.

15. The method of any of claims 1-14, wherein the adenoviral vectors are deficient in one or more gene functions of the E3 region of the adenoviral genome.

16. The method of any of claims 1-15, wherein at least one HIV antigen is selected from the group consisting of an HIV Gag protein, HIV Pol protein, HIV Env protein, HIV Tat protein, HIV Reverse Transcriptase (RT) protein, HIV Vif protein, HIV Vpr protein, HIV Vpu protein, HIV Vpo protein, HIV Integrase protein, HIV Nef protein, and a fusion protein comprising all or part of an HIV Gag protein, HIV Pol protein, or HIV Env protein.

17. The method of claim 16, wherein at least one HIV antigen is HIV gp140 or gp140dv12.

18. The method of claim 16, wherein at least one HIV antigen is a fusion protein that comprises all or part of an HIV Gag protein and all or part of an HIV Pol protein.

19. The method of any of claims 1-18, wherein the HIV antigens comprise at least one member selected from the group consisting of an HIV clade A antigen, HIV clade B antigen, HIV clade C antigen, and HIV clade MN antigen.

20. The method of claim 19, wherein the HIV antigens comprise at least two members selected from the group consisting of an HIV clade A antigen, HIV clade B antigen, HIV clade C antigen, and HIV clade MN antigen.

21. The method of claim 20, wherein the adenoviral vectors encode three or more different HIV antigens, and the HIV antigens comprise at least three members selected from the group consisting of an HIV clade A antigen, HIV clade B antigen, WV clade C antigen, and V clade MN antigen.

22. The method of any of claims 1-21, wherein the adenoviral vector composition is administered as part of a pharmaceutical composition comprising a pharmaceutically acceptable carrier.

23. The method of claim 22, wherein the pharmaceutical composition is administered in two or more doses.

24. The method of claim 22 or 23, wherein the pharmaceutical composition is administered in a dose comprising 1×10⁸ to 1×10¹² particle units (pu) adenoviral vector.

25. The method of claim 24, wherein the pharmaceutical composition is administered in a dose comprising 1×10⁸ to 1×10¹⁰ pu adenoviral vector.

26. The method of claim 24, wherein the pharmaceutical composition is administered in a dose comprising 1×10⁹ to 1×10¹¹ pu adenoviral vector.

27. The method of claim 24, wherein the pharmaceutical composition is administered in a dose comprising 1×10¹⁰ to 1×10¹² pu adenoviral vector.

28. The method of any of claims 1-27, wherein the adenoviral vector composition comprises (a) an adenoviral vector comprising a nucleic acid encoding a fusion protein comprising an HIV clade B Gag protein and Pol protein, (b) an adenoviral vector comprising a nucleic acid encoding an HIV clade A Env protein, (c) an adenoviral vector comprising a nucleic acid encoding an HIV clade B Env protein, and (d) an adenoviral vector comprising a nucleic acid encoding an HIV clade C Env protein.

29. The method of claim 28, wherein the fusion protein comprising an HIV clade B Gag protein and Pol protein is encoded by a nucleic acid sequence that further encodes HIV Protease, Reverse Transcriptase (RT), and Integrase proteins, and wherein the nucleic acid molecule comprises one or more point mutations, which point mutations render the Protease, RT, and Integrase proteins non-functional.

30. The method of claim 28, wherein the Env protein is gp140 or gp140dv12.

31. The method of claim 1, wherein the adenoviral vector composition comprises four adenoviral vectors having the nucleic acid sequences of SEQ D NO: 4, SEQ ID NO: 5, SEQ U) NO: 6, and SEQ ID NO: 7, respectively.

32. An adenoviral vector composition comprising (a) an adenoviral vector comprising a nucleic acid encoding a fusion protein comprising an HIV clade B Gag protein and Pol protein, (b) an adenoviral vector comprising a nucleic acid encoding an HIV clade A Env protein, (c) an adenoviral vector comprising a nucleic acid encoding an HIV clade B Env protein, and (d) an adenoviral vector comprising a nucleic acid encoding an HIV clade C Env protein.

33. The adenoviral vector composition of claim 32, wherein the fusion protein comprising an HIV clade B Gag protein and Pol protein is encoded by a nucleic acid sequence that further encodes HIV Protease, Reverse Transcriptase (RT), and Integrase proteins, and wherein the nucleic acid molecule comprises one or more point mutations, which point mutations render the Protease, RT, and Integrase proteins non-functional.

34. The adenoviral vector composition of claim 32, wherein the Env protein is gp140 or gp140dv12.

35. The adenoviral vector composition of claim 32, wherein (a), (b), (c), and (d) have the nucleic acid sequences of SEQ ID NO: 4, SEQ ID NO: 5, SEQ ID NO: 6, and SEQ ID NO: 7, respectively.

36. The adenoviral vector composition of any of claims 32-35, wherein the adenoviral vectors are replication-deficient.

37. The adenoviral vector composition of claim 36, wherein the adenoviral vectors are deficient in one or more essential gene functions of the E1 region of the adenoviral genome.

38. The adenoviral vector composition of claim 36 or 37, wherein the adenoviral vectors are deficient in one or more essential gene functions of the E4 region of the adenoviral genome.

39. The adenoviral vector composition of any of claims 32-38, wherein the adenoviral vectors are deficient in one or more gene functions of the E3 region of the adenoviral genome.

40. The adenoviral vector composition of any of claims 32-39, wherein (a), (b), (c), and (d) are present in the composition in a ratio of 3:1:1:1 by weight.

41. A pharmaceutical composition comprising the adenoviral vector composition of any of claims 32-40 and a pharmaceutically acceptable carrier.

42. The pharmaceutical composition of claim 41, wherein (a), (b), (c), and (d) have the nucleic acid sequences of SEQ ID NO: 4, SEQ ID NO: 5, SEQ ID NO: 6, and SEQ ID NO: 7, respectively.

43. The pharmaceutical composition of claim 41 or 42, comprising about 1×10⁸ to 1×10¹² particle units (pu) adenoviral vector.

44. The pharmaceutical composition of claim 43, comprising about 1×10⁸ to 1×10¹⁰ pu adenoviral vector.

45. The pharmaceutical composition of claim 43, comprising about 1×10⁹ to 1×10¹¹ pu adenoviral vector.

46. The pharmaceutical composition of claim 43, comprising about 1×10⁹ to 1×10¹² pu adenoviral vector.
